SEQUENCE LISTING (13 sequences)

SEQ ID NO: 1 - protein, 8 aa, Moraxella catarrhalis
NKYAGKGY

SEQ ID NO: 2 - protein, 8 aa, Moraxella catarrhalis
DKYAGKGY

SEQ ID NO: 3 - protein, 8 aa, Moraxella catarrhalis
KDYPGQGY

SEQ ID NO: 4 - protein, 8 aa, Moraxella catarrhalis
KKYPGQGY

SEQ ID NO: 5 - protein, 18 aa, Moraxella catarrhalis
QAELDNKYAGKGYKLGSK

SEQ ID NO: 6 - protein, 18 aa, Moraxella catarrhalis
QAELDDKYAGKGYKLGSK

SEQ ID NO: 7 - protein, 18 aa, Moraxella catarrhalis
LAELNKDYPGQGYKLGKK

SEQ ID NO: 8 - DNA, 39 bp, Moraxella catarrhalis
ggatccgccaccatggctaataagtttcaattattaccg

SEQ ID NO: 9 - DNA, 24 bp, Moraxella catarrhalis
gaattccccataaaaagaacaccc

SEQ ID NO: 10 - DNA, 18 bp, Moraxella catarrhalis
caccaagctctacactgg

SEQ ID NO: 11 - DNA, 18 bp, Moraxella catarrhalis
caccaagctctacactgg

SEQ ID NO: 12 - DNA, 18 bp, Moraxella catarrhalis
ctgacattggcggtgagt

SEQ ID NO: 13 - DNA, 17 bp, Moraxella catarrhalis
gtgcttgagtgttcagt

(19) United States
(12) Patent Application Publication
(10) Pub. No.: US 2006/0147470 A1
(43) Pub. Date: Jul. 6, 2006

(54) Cross-protective epitopes of moraxella catarrhalis and use thereof

(76) Inventors: Dai-Fang Liu, New City, NY (US); John C. McMichael, Rochester, NY (US); Steven Morris Baker, Highland Mills, NY (US); Leah Diane Fletcher, Geneseo, NY (US)

Correspondence Address: WYETH;PATENT LAW GROUP, 5 GIRALDA FARMS, MADISON, NJ, 07940, US

(21) Appl. No.: 10/544,048

(22) PCT Filed: Jan. 27, 2004
(86) PCT No.: PCT/US04/02383

§ 371 Date: Mar. 7, 2006

(30) Foreign Application Priority Data
Jan. 30, 2003 (US) 60443600

(51) Int. Cl. A61K 39/02

(52) U.S. Cl. 424/234.1; 424/190.1

(57) ABSTRACT

The present invention broadly relates to immunogenic compositions for administering to a host susceptible to Moraxella catarrhalis infection. More particularly, the invention is directed to the identification of cross-reactive epitope sequences of the CopB protein of Moraxella catarrhalis.

### FIELD OF THE INVENTION

The present invention generally relates to the fields of microbiology, clinical bacteriology and immunology. More particularly, the invention relates to the identification of novel cross-reactive epitope sequences of the CopB protein of Moraxella catarrhalis.

### BACKGROUND OF THE INVENTION

Moraxella catarrhalis, along with Haemophilus influenzae and Streptococcus pneumoniae, are the three major bacterial causes of otitis media (OM). Among young children, otitis media is the most common reason for visits to pediatricians, and otitis media with effusion can lead to developmental problems as late as the teens (Bennett et al., 2001). It is estimated that Moraxella catarrhalis accounts for up to 23% of all the otitis media cases (Murphy, 1996). In addition, Moraxella catarrhalis is frequently associated with respiratory infections in older adults, especially exacerbations of chronic obstructive pulmonary disease (Sethi and Murphy, 2001).

Thus, immunogenic compositions to prevent Moraxella catarrhalis infections would significantly benefit the health of these populations. In fact, a 1989 Consensus Report concluded that prevention of otitis media is an important health care goal due to both its occurrence in infants and children, as well as certain populations of all age groups (Anonymous, “Consensus”, Pediatr. Infect. Dis. J., 8:594-597, 1989). The total financial burden of otitis media has been estimated to be at least $2.5 billion annually. Vaccines were identified in the Consensus Report as the most desired approach to prevent otitis media.

The CopB protein of Moraxella catarrhalis is one of several promising candidate antigens for inclusion in immunogenic compositions. This 80 kDa integral outer membrane protein mediates iron acquisition and is essential for the survival of the bacterium in vivo (Helminen et al., 1993a). It has been reported that the copB gene is present in every isolate examined (Bootsma et al., 2000) and is relatively well conserved from isolate to isolate (Sethi et al., 1997; Helminen et al., 1993b). The CopB protein shares homology with the FetA proteins of the Neisselia (formerly known as the FrpB proteins) which act as receptors for the enterobactin siderophore (Aebi et al, 1996; Campagnari et al., 1994; Carson et aL, 1999). The main reason CopB is considered a potential candidate antigen is that a CopB specific monoclonal antibody (MAb) 10F3 exhibits complement dependent bactericidal activity (Aebi et al, 1998), and in a pulmonary colonization challenge model, passive immunization of mice with 10F3 promotes bacterial clearance (Helminen et at., 1993b).

The only published information related to CopB serology has come from studies of MAb reactivities. Helminen et al showed that MAb 10F3 recognized about 70% of the Moraxella catarrhalis isolates (Helminen et al, 1993b). A similar pattern of reactivity was also reported by Sethi et aL using a different set of MAbs (Sethi et al., 1997), and a report from that same laboratory suggested that CopB might have an immuno-dominant epitope (Ameen et al, 1998). Mapping has revealed that the epitope domain for MAb 10F3 occurs between amino acid residues 295 and 302 of the CopB protein, and that there is a single amino acid difference between the epitope of the 10F3 positive isolate 035E and the epitope of the 10F3 negative isolate TTA24 (Aebi et al., 1998; International Application WO 98/06851).

As stated above, the CopB outer membrane protein of Moraxella catarrhalis is under investigation as a candidate immunogen. An effective immunogen needs to elicit cross protection against most, if not all strains of Moraxella catarrhalis occurring in the population. It is therefor highly desirable in the art to identify one or more CopB epitopes that confer protection (i.e., cross-protection) against a high percentage of Moraxella catarrhalis strains. It is contemplated that such CopB cross-reactive epitopes will be therapeutically useful as immunogenic compositions for administration to a host susceptible to Moraxella catarrhalis infection.

### SUMMARY OF THE INVENTION

The present invention broadly relates to immunogenic compositions for administering to a host susceptible to Moraxella catarrhalis infection. More particularly, the invention is directed to the identification of cross-reactive epitope sequences of the CopB protein of Moraxella catarrhalis.

In particular, the invention makes use of the finding of two novel Moraxella catarrhalis CopB serogroups: Serogroup III, which has the CopB epitope domain of SEQ ID NO:3; and serogroup IV, which has the CopB epitope domain of SEQ ID NO:4.

In certain embodiments, the invention is directed to an immunogenic composition comprising Moraxella catarrhalis polypeptide fragments, wherein the fragments comprise amino acid sequences of SEQ ID NO:1, SEQ ID NO:2 and SEQ ID NO:3. In another embodiment, the invention provides an immunogenic composition comprising Moraxella catarrhalis polypeptide fragments, wherein the fragments comprise amino acid sequences of SEQ ID NO:1, SEQ ID NO:2 and SEQ ID NO:4. In yet another embodiment, the invention provides an immunogenic composition comprising Moraxella catarrhalis polypeptide fragments, wherein the fragments comprise amino acid sequences of SEQ ID NO:1, SEQ ID NO:2, SEQ ID NO:3 and SEQ ID NO:4. In one embodiment, the fragments are further defined as CopB epitopes. In another embodiment, the invention provides an immunogenic composition comprising Moraxella catarrhalis polypeptide fragments, wherein the fragments comprise amino acid sequences of SEQ ID NO:3 and/or SEQ ID NO:4. In certain other embodiments, SEQ ID NO:3 is defined as an amino acid sequence comprised within a CopB polypeptide of Moraxella catarrhalis isolate 430:345, Moraxella catarrhalis isolate 046E or Moraxella catarrhalis isolate 56. In yet other embodiments, SEQ ID NO:4 is defined as an amino acid sequence comprised within a CopB polypeptide of Moraxella catarrhalis isolate 4608.1.

In certain other embodiments, the fragments are covalently associated to a carrier protein (conjugated) and may further comprise one or more adjuvants. In a preferred embodiment, the immunogenic compositions, when administered to a mammalian host in an immunogenic amount, protects the host against greater than 75% of Moraxella catarrhalis strains.

In one embodiment, the invention is directed to an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:3. In certain embodiments, the immunogenic composition further comprises a fourth polypeptide comprising at least the amino acid sequence of SEQ ID NO:4. In another embodiment, the invention is directed to an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:4. In still other embodiments, the invention is directed to an immunogenic composition comprising at least two Moraxella catarrhalis polypeptides, wherein the first polypeptide comprises at least the amino acid sequence of SEQ ID NO:3 and the second polypeptide comprises at least the amino acid sequence of SEQ ID NO:4. In still other embodiments, the invention is directed to an immunogenic composition comprising a Moraxella catarrhalis polypeptide comprising the amino acid sequence of SEQ ID NO:3. In still other embodiments, the invention is directed to an immunogenic composition comprising a Moraxella catarrhals polypeptide comprising the amino acid sequence of SEQ ID NO:4. In certain embodiments, the immunogenic composition further comprising a fourth polypeptide comprises at least the amino acid sequence of SEQ ID NO:3. In yet other embodiments, the polypeptides are further defined as CopB polypeptides. In a preferred embodiment, SEQ ID NO:3 is further defined as an amino acid sequence comprised within a CopB polypeptide of Moraxella catarrhalis isolate 430:345, Moraxella catarrhalls isolate 046E or Moraxella catarrhalis isolate 56. In still other preferred embodiments, SEQ ID NO:4 is further defined as an amino acid sequence comprised within a CopB polypeptide of Moraxella catarrhalis isolate 4608.1. In other embodiments, the polypeptides are covalently associated to a carrier protein and may further comprise one or more adjuvants. In certain embodiments, the immunogenic composition, when administered to a mammalian host in an immunogenic amount, protects the host against greater than 75% of Moraxella catarrhalis strains.

The invention is directed in one particular embodiment, to an isolated polypeptide comprising a plurality of covalently linked Moraxella catarrhals CopB epitope fragments, wherein the polypeptide comprises at least a fragment comprising an amino acid sequence of SEQ ID NO:1, a fragment comprising an amino acid sequence of SEQ ID NO:2 and a fragment comprising an amino acid sequence of SEQ ID NO:3. In other embodiments, the invention is directed to an isolated polypeptide comprising a plurality of covalently linked Moraxella catarrhalis CopB epitope fragments, wherein the polypeptide comprises at least a fragment comprising an amino acid sequence of SEQ ID NO:1, a fragment comprising an amino acid sequence of SEQ ID NO:2 and a fragment comprising an amino acid sequence of SEQ ID NO:4. In yet other embodiments, the invention is directed to an isolated polypeptide comprising a plurality of covalently linked Moraxella catarrhalis CopB epitope fragments, wherein the polypeptide comprises at least a fragment comprising an amino acid sequence of SEQ ID NO:1, a fragment comprising an amino acid sequence of SEQ ID NO:2, a fragment comprising an amino acid sequence of SEQ ID NO:3 and a fragment comprising an amino acid sequence of SEQ ID NO:4. In one embodiment, the invention is directed to a polynucleotide encoding a polypeptide comprising a plurality of covalently linked Moraxella catarrhalis CopB epitope fragments. In another embodiment, the invention provides an expression vector comprising a polynucleotide encoding a polypeptide comprising a plurality of covalently linked Moraxella catarrhalis CopB epitope fragments. In still other embodiments, the invention provides a host cell transformed, transfected or infected with an expression vector comprising a polynucleotide encoding a polypeptide comprising a plurality of covalently linked Moraxella catarrhalis CopB epitope fragments.

In certain embodiments, the invention is directed to a method of immunizing a host against Moraxella catarrhalis infection, the method comprising administering to the host an immunogenic composition comprising Moraxella catarrhalis polypeptide fragments, wherein the fragments comprise amino acid sequences of SEQ ID NO:1, SEQ ID NO:2 and SEQ ID NO:3, wherein the method may further comprise a fragment comprising an amino acid sequence of SEQ ID NO:4.

In still other embodiments, the invention is directed to a method of immunizing a host against Moraxella catarrhalis infection, the method comprising administering to the host an immunogenic composition comprising Moraxella catarrhalis polypeptide fragments, wherein the fragments comprise amino acid sequences of SEQ ID NO:1, SEQ ID NO:2 and SEQ ID NO:4, wherein the method may further comprise a fragment comprising an amino acid sequence of SEQ ID NO:3.

In still another embodiment, the invention is directed to a method of immunizing a host against Moraxella catarrhalis infection, the method comprising administering to the host an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:3, wherein the method may further comprise a fourth polypeptide comprising at least an amino acid sequence of SEQ ID NO:4.

In yet another embodiment, the invention is directed to a method of immunizing a host against Moraxella catarrhalis infection, the method comprising administering to the host an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:4, wherein the method may further comprise a fourth polypeptide comprising at least an amino acid sequence of SEQ ID NO:3.

In further embodiments, the invention is directed to a method of immunizing a host against Moraxella catarrhalis infection, the method comprising administering to the host an immunogenic composition comprising a plurality of covalently linked Moraxella catarrhalis CopB epitope fragments, wherein the plurality comprises at least a fragment comprising an amino acid sequence of SEQ ID NO:1, a fragment comprising an amino acid sequence of SEQ ID NO:2 and a fragment comprising an amino acid sequence of SEQ ID NO:3, wherein the method may further comprise a fourth fragment comprising at least an amino acid sequence of SEQ ID NO:4.

In certain embodiments, the invention is directed to a method of immunizing a host against Moraxella catarrhalis infection, the method comprising administering to the host an immunogenic composition comprising a plurality of covalently linked Moraxella catarrhalis CopB epitope fragments, wherein the plurality comprises at least a fragment comprising an amino acid sequence of SEQ ID NO:1, a fragment comprising an amino acid sequence of SEQ ID NO:2 and a fragment comprising an amino acid sequence of SEQ ID NO:4, wherein the method may further comprise a fourth fragment comprising at least an amino acid sequence of SEQ ID NO:3.

In particular embodiments, the polypeptides in any of the above methods may be conjugated to an antigen carrier protein, may comprise additional antigens, and/or may comprise one or more adjuvants.

Other features and advantages of the invention will be apparent from the following detailed description, from the preferred embodiments thereof, and from the claims.

### BRIEF DESCRIPTION OF THE FIGURES

FIG. 1 shows an alignment of the amino acid sequences of the CopB proteins from M. catarrhalis isolates 035E, TTA24, 430:345, 218:038, 417:082 and 4608.1. The shaded area indicates the surface exposed epitope defined by MAb 10F3. The boxed letters reflect amino acid differences among the isolates.

### DETAILED DESCRIPTION OF THE INVENTION

The invention described hereinafter, addresses the need in the art for effective immunogenic compositions for administering to a mammalian host (e.g., a human) susceptible to Moraxella catarrhalis infection. An antigen or immunogen is typically defined on the basis of immunogenicity. Immunogenicity is defined as the ability to induce a humoral and/or cell-mediated immune response. Thus, the terms antigen or immunogen, as defined hereinafter, are molecules possessing the ability to induce a humoral and/or cell-mediated immune response.

In one particular embodiment, the present invention has identified two novel Moraxella catarrhalis CopB serogroups (hereinafter, serogroup III and serogroup IV). The representative isolates of serogroup III are Moraxella catarrhalis isolate 430:345, isolate 56 and isolate 046E. The representative isolate of serogroup IV is Moraxella catarrhalis isolate 4608.1. Serogroups III and IV were identified during the characterization of Moraxella catarrhalis isolates that do not bind monoclonal antibody 10F3 (i.e., 10F3 negative isolates). It is known in the art, that the monoclonal antibody 10F3 (hereinafter, 10F3 or MAb 10F3) recognizes about 70% of the Moraxella catarrhalis isolates (Helminen et al., 1993b) and it has been suggested that CopB might have an immuno-dominant epitope (Ameen et al., 1998). Protein mapping has revealed that the epitope domain for MAb 10F3 occurs between amino acid residues 295 and 302 of the CopB protein. As defined herein, a CopB epitope domain recognized by 10F3 has an 8 residue amino acid sequence of NKYAGKGY (SEQ ID NO:1), wherein SEQ ID NO:1 is comprised within a Moraxella catarrhalis CopB serogroup I protein from about amino acid residue 295 to about amino acid residue 302.

The copB gene from isolate 430:345 (a 10F3 negative isolate) was sequenced in the region corresponding to the 10F3 epitope domain and a four amino acid difference was detected from that of the 035E isolate (a 10F3 positive isolate; See, Table 1 and FIG. 2). Similarly, the copB gene from isolate 4608.1 (a 10F3 negative isolate) was sequenced in the region corresponding to the 10F3 epitope and a three amino acid difference was detected from that of the O35E isolate (See, Table 1)

As defined herein, a “10F3 positive” isolate is a Moraxella catarrhalis isolate that binds 10F3. As defined herein, a “10F3 negative isolate” is a Moraxella catarrhalis isolate that does not bind 10F3.

As defined herein, a “CopB epitope” or an “epitope domain” of a Moraxella catarrhalis CopB protein relates to the 8 residue amino acid sequence NKYAGKGY (SEQ ID NO:1). More specifically, as defined herein, a CopB epitope of Moraxella catarrhalis serogroup I has an amino acid sequence of NKYAGKGY (SEQ ID NO:1). Representative isolates of serogroup I are isolates O35E, O12E and ATCC25240. As defined herein, a CopB epitope of Moraxella catarrhalis serogroup II has an amino acid sequence of DKYAGKGY (SEQ ID NO:2), wherein the bold residue indicates a different residue from that of SEQ ID NO:1. A representative isolate of serogroup II is isolate TTA24. As defined herein, a CopB epitope of the newly identified Moraxella catarrhalis serogroup III has an amino acid sequence of KDYPGOGY (SEQ ID NO:3), wherein bold residues indicate a different residue from that of SEQ ID NO:1. Representative isolates of serogroup III are isolates 430:345, O46E and 56. As defined herein, a CopB epitope of the newly identified Moraxella catarrhalis serogroup IV has an amino acid sequence of KKYPGQGY (SEQ ID NO:4), wherein bold residues indicate a different residue from that of SEQ ID NO:1. A representative isolate of serogroup IV is isolate 4608.1.

Thus, the identification of the novel CopB serogroups (ie., serogroup III and IV) of the present invention will be particularly useful in developing immunogens and compositions thereof, that confer a high percentage of protection against the majority (e.g., >75%) of Moraxella catarrhalis strains.

TABLE 1
CopB Serogroup with Epitope Sequence

| | Prototypic | Genebank | Epitope |
|---|---|---|---|
| Serogroup | isolates | accession no. | sequences^{a} |
| CopB-I | 035E | L12346 | NKYAGKGY |
| | 012E | U69981 | (SEQ ID NO:1) |
| | ATCC25240 | U83900 | |
| CopB-II | TTA24 | U69980 | DKYAGKGY |
| | | | (SEQ ID NO:2) |
| CopB-III | 430:345 | TBD^{c} | KDYPGQGY |
| | 046E | U69982 | (SEQ ID NO:3) |
| | 56 | U83901 | |
| CopB-IV | 4608.1 | TBD | KKYPGQGY |
| | | | (SEQ ID NO:4) |

^{a}The amino acid sequence of the domain corresponding to the 10F3 epitope was deduced from the DNA sequence.
^{b}TBD = to be deposited.

### A. COPB POLYPEPTIDES AND FRAGMENTS THEREOF

In particular embodiments, the present invention provides immunogens comprising the Moraxella catarrhalis CopB epitope. In one particular embodiment, these immunogens are used in the preparation of immunogenic compositions for immunizing a mammalian host against Moraxella catarrhalis infections. In preferred embodiments, these immunogens confer protection (ie., cross-protection) against a high percentage of heterologous Moraxella catarrhalis strains.

Thus, in one embodiment, the invention is directed to an immunogenic composition comprising Moraxella catarrhalis polypeptide fragments, wherein the fragments comprise amino acid sequences of SEQ ID NO:1, SEQ ID NO:2 and SEQ ID NO:3. In another embodiment an immunogenic composition comprises Moraxella catarrhalis polypeptide fragments, wherein the fragments comprise amino acid sequences of SEQ ID NO:1, SEQ ID NO:2 and SEQ ID NO:4. In yet another embodiment, an immunogenic composition comprises Moraxella catarrhalis polypeptide fragments, wherein the fragments comprise amino acid sequences of SEQ ID NO:1, SEQ ID NO:2, SEQ ID NO:3 and SEQ ID NO:4.

In certain other embodiments, the present invention is directed to an Immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:3 or at least the amino acid sequence of SEQ ID NO:4.

Certain other embodiments are directed to a plurality of covalently linked CopB epitope fragments. Thus, in one embodiment, the invention provides an isolated polypeptide comprising a plurality of covalently linked Moraxella catarrhalis CopB epitope fragments, wherein the polypeptide comprises at least a fragment comprising an amino acid sequence of SEQ ID NO:1, a fragment comprising an amino acid sequence of SEQ ID NO:2 and a fragment comprising an amino acid sequence of SEQ ID NO:3 and/or a fragment comprising an amino acid sequence of SEQ ID NO:4. As described below, each fragment is conjugated to a carrier protein.

Preferably, the full length CopB polypeptides of the invention are recombinant polypeptides. The CopB polypeptide fragments of the invention may be recombinantly expressed or prepared via peptide synthesis methods known in the art (Barany et al., 1987; U.S. Pat. No. 5,258,454).

A biological equivalent or variant of a CopB polypeptide according to the present invention encompasses a polypeptide that contains substantial homology to a CopB polypeptide, as long as the CopB epitope domain defined by SEQ ID NOs:1-4 is conserved within the polypeptide sequence. Biological equivalents or variants also include those polypeptides where even the CopB epitope domain defined by SEQ ID NOs:1-4 is modified, so long as the polypeptide maintains the ability to elicit an immunogenic response via the modified CopB epitope domain

Generally, functional biological equivalents or variants are naturally occurring amino acid sequence variants of a CopB polypeptide, wherein the polypeptide maintains the ability to elicit an immunogenic response via the CopB epitope domain.

Modifications and changes can be made in the structure of a polypeptide of the present invention and still obtain a molecule having CopB epitope immunogenic properties. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those that are within ±1 are particularly preferred, and those within ±0.5 are even more particularly preferred.

Substitution of like amino acids can also be made on the basis of hydrophilicity, particularly where the biological functional equivalent polypeptide or polypeptide fragment, is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−4); alanine (−0.5); histidine (−0.5); cysteine (−1.0); methionine (−1.3); valine (−1.5); leucine (−1.8); isoleucine (−1.8); tyrosine (−2.3); phenylalanine (±2.5); tryptophan (±3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 2, below). The present invention thus contemplates functional or biological equivalents of a CopB polypeptide as set forth above.

TABLE 2
AMINO ACID SUBSTITUTIONS

| | Exemplary Residue |
|---|---|
| Original Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

A CopB polypeptide or polypeptide fragment of the present invention is understood to be any CopB polypeptide comprising substantial sequence similarity, structural similarity and/or functional similarity to a CopB polypeptide comprising an epitope domain of SEQ ID NO1, SEQ ID NO:2, SEQ ID NO:3 or SEQ ID NO:4. In addition, a CopB polypeptide of the invention is not limited to a particular source.

It is contemplated in the present invention, that a CopB polypeptide may advantageously be cleaved into fragments for use in further structural or functional analysis, or in the generation of reagents such as CopB-related polypeptides and CopB-specific antibodies. This can be accomplished by treating purified or unpurified CopB polypeptides with a peptidase such as endoproteinase glu-C (Roche Diagnostics Corporation, Indianapolis, Ind.). Treatment with CNBr is another method by which CopB peptide fragments may be produced from natural CopB polypeptides. Recombinant techniques also can be used to produce one or more specific fragments of a CopB polypeptide, either alone or in covalent linkage to each other.

In addition, the inventors also contemplate that compounds sterically similar to a particular CopB polypeptide immunogen may be formulated to mimic the key portions of the CopB epitope domain, called peptidomimetics or peptide mimetics. Mimetics are peptide-containing molecules which mimic elements of protein secondary structure. (see, e.g. Johnson et al., 1993; U.S. Pat. No. 5,424,334; U.S. Pat. No. 4,992,463 and U.S. Pat. No. 5,552,431). The underlying rationale behind the use of peptide mimetics is that the peptide backbone of proteins exists chiefly to orient amino acid side chains in such a way as to facilitate molecular interactions, such as those of receptor and ligand.

Successful applications of the peptide mimetic concept have thus far focused on mimetics of β-turns within proteins. Likely β-turn structures within CopB can be predicted by computer-based algorithms known in the art. Once the component amino acids of the turn are determined, mimetics can be constructed to achieve a similar spatial orientation of the essential elements of the amino acid side chains, as discussed in Johnson et al., 1993.

As described above, CopB polypeptide fragments of the invention (e.g., SEQ ID Nos: 1-4) are of particular utility as immunogens. A fragment, which is produced by chemical synthesis or recombinant expression methodologies, is a polypeptide having an amino acid sequence that entirely is the same as part, but not all, of the amino acid sequence. The fragment can comprise, for example, at least 7 or more (e.g., 7, 8, 10, 12, 14, 16, 18, 20, or more) contiguous amino acids of an amino acid sequence of SEQ ID NO:1, SEQ ID NO:2, SEQ ID NO:3 or SEQ ID NO:4. Fragments may be “freestanding” or comprised within a larger polypeptide of which they form a part or region, most preferably as a single, continuous region. In one embodiment, the fragments comprise the epitope domain (e.g., SEQ ID NO:1) of the mature CopB polypeptide sequence. In another embodiment, a plurality of these epitope domains (e.g., SEQ ID NO:1, SEQ ID NO:2, SEQ ID NO:3 and/or SEQ ID NO:4) are covalently linked to form a larger polypeptide entity. For example, a plurality of covalently linked epitopes of the invention comprises a tandem repeat of the following fragment sequences; (SEQ ID NO:1-SEQ ID NO:2-SEQ ID NO:3 and/or -SEQ ID NO:4)ₙ or any order thereof and/or combination thereof. This is only an example and should not be construed as limiting.

“Fusion protein” refers to a protein or polypeptide encoded by two, often unrelated, fused genes or fragments thereof. For example, fusion proteins or polypeptides comprising various portions of constant region of immunoglobulin molecules together with another human protein or part thereof have been described. In many cases, employing an immunoglobulin Fc region as a part of a fusion protein or polypeptide is advantageous for use in therapy and diagnosis resulting in, for example, improved pharmacokinetic properties. On the other hand, for some uses it is desirable to delete the Fc part after the fusion protein or polypeptide has been expressed, detected and purified.

In particular embodiments, the invention is directed to compositions and methods of use comprising a CopB polypeptide conjugated to an antigen carrier protein or a bacterial cell, either as a recombinant fusion polypeptide or through chemical attachment (i.e., conjugation). Means for conjugating a polypeptide to a carrier protein are well known in the art and include glutaraldehyde, m-maleimidobencoyl-N-hydroxysuccinimide ester, carbodiimide and bis-biazotized benzidine.

Exemplary conventional protein carriers include, without limitation, E. coli DnaK protein, galactokinase (galk, which catalyzes the first step of galactose metabolism in bacteria), ubiquitin, α-mating factor, β-galactosidase, and influenza NS-1 protein. Toxoids (ie., the sequence which encodes the naturally occurring toxin, with sufficient modifications to eliminate its toxic activity) such as diphtheria toxoid and tetanus toxoid, their respective toxins, and any mutant forms of these proteins, may also be employed as carriers. An exemplary carrier protein is diphtheria toxin CRM₁₉₇ (a non-toxic form of diphtheria toxin, see U.S. Pat. No. 5,614,382). Other carriers include exotoxin A of Pseudomonas, heat labile toxin of E. coli, Vibrio cholera and rotaviral particles (including rotavirus and VP6 particles). Alternatively, a fragment or epitope of the carrier protein or other immunogenic protein may be used. For example, a hapten may be coupled to a T cell epitope of a bacterial toxin. See U.S. Pat. No. 5,785,973. Similarly a variety of bacterial heat shock proteins, e.g., mycobacterial hsp-70 may be used. Glutathione-S-transferase (GST) is another useful carrier. One of skill in the art can readily select an appropriate carrier for use in this context.

In certain embodiments, the invention is directed to compositions and methods of use comprising a plurality of covalently linked CopB epitope fragments. Due to the small size of the CopB epitope domains, it is contemplated that a single fragment may not always provide optimal immunogencity. Thus, in certain embodiments, the invention provides recombinant CopB polypeptides containing tandem (i.e., a plurality) CopB epitope fragments of different CopB strains expressed by gene fusions of the appropriate epitope region of several copB genes. Thus, a plurality or tandem of CopB epitope fragments is any number and/or any order of CopB epitope fragments covalently linked together or to a carrier protein. In a preferred embodiment, a recombinant polypeptide comprising a plurality of CopB epitope fragments includes at least a CopB epitope domain from serogroup I, serogroup II and serogroup III (e.g., SEQ ID NO:1, SEQ ID NO:2 and SEQ ID NO:3). In another preferred embodiment, a recombinant polypeptide comprising a plurality of CopB epitope fragments includes at least a CopB epitope domain from serogroup I, serogroup II and serogroup IV. In still another embodiment a recombinant polypeptide comprising a plurality of CopB epitope fragments includes at least a CopB epitope domain from serogroup I, serogroup II, serogroup III and serogroup IV.

Accordingly, such tandem molecules can be engineered to maintain proper structure at points of junction and to be large enough to be immunogenic and to express an array of epitopes that cross-react with a wide spectrum of Moraxella catarrhalis strains. Alternatively, individual recombinantly produced or synthesized fragments may be attached by chemical means to form a plurality of covalently linked fragments.

### B. MORAXELLA CATARRHALIS POLYNUCLEOTIDES

Isolated and purified Moraxella catarrhalis polynucleotides of the present invention are contemplated for use in the production of CopB polypeptide and polypeptide fragments. More specifically, in certain embodiments, the polynucleotides encode CopB polypeptide fragments or fusion polypeptides, particularly the CopB epitopes of SEQ ID Nos: 1-4.

In particular embodiments, a polynucleotide of the present invention is a DNA molecule, wherein the DNA may be genomic DNA, chromosomal DNA, plasmid DNA or cDNA. In a preferred embodiment, a polynucleotide of the present invention is a recombinant polynucleotide, which encodes a CopB polypeptide (e.g., a CopB epitope domain). In a preferred embodiment, a polynucleotide encoding a CopB polypeptide is comprised in a plasmid vector and expressed in a prokaryotic host cell.

As used hereinafter, the term “polynucleotide” means a sequence of nucleotides connected by phosphodiester linkages. Polynucleotides are presented hereinafter from the 5′ to the 3′ direction. A polynucleotide of the present invention can comprise from about 10 to about several hundred thousand base pairs. Preferably, a polynucleotide comprises from about 10 to about 3,000 base pairs. Preferred lengths of particular polynucleotide are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated hereinafter by a single letter code: adenine (A), guanine (G), thymine (T), cytosine (C), inosine (I) and uracil (U).

“Isolated” means altered “by the hand of man” from the natural state. An “isolated” composition or substance is one that has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed hereinafter.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived. For example, in various embodiments, the isolated CopB nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. However, the CopB nucleic acid molecule can be fused to other protein encoding or regulatory sequences and still be considered isolated.

CopB polynucleotides of the present invention are obtained using standard cloning and screening techniques, from a cDNA library derived from mRNA. Polynucleotides of the invention are also obtained from natural sources such as genomic DNA libraries (e.g., a Moraxella catarrhalis library) or are synthesized using well known and commercially available techniques.

Orthologues and allelic variants of the CopB polynucleotides are readily identified using methods well known in the art. Allelic variants and orthologues of the polynucleotides comprise a nucleotide sequence that is typically at least about 70-75%, more typically at least about 80-85%, and most typically at least about 90-95% or more homologous to the nucleotide sequences shown in SEQ ID NOs:1-4, or a fragment of these nucleotide sequences. Such nucleic acid molecules are readily identified as being able to hybridize, preferably under stringent conditions, to the nucleotide sequence shown in SEQ ID NOs:1-4, or a fragment of these nucleotide sequences.

In certain preferred embodiments, a polynucleotide of the invention comprises only a fragment of the coding region of a CopB polynucleotide or gene, such as a fragment encoding the CopB epitope domains, e.g., a polypeptide fragment having an amino acid sequence of one of SEQ ID NOs:1-4.

When the CopB polynucleotides of the invention are used for the recombinant production of CopB polypeptides or polypeptide fragments, the polynucleotide may include the coding sequence for the mature polypeptide, by itself, or the coding sequence for the mature polypeptide in reading frame with other coding sequences, such as those encoding a leader or secretory sequence, a pre-, a pro- a prepro-protein sequence, or other fusion peptide portions. For example, a marker sequence which facilitates purification of the fused polypeptide can be linked to the coding sequence (see Gentz et al., 1989, incorporated by reference hereinafter in its entirety). Thus, contemplated in the present invention is the preparation of polynucleotides encoding fusion polypeptides permitting His-tag purification of expression products. The polynucleotide may also contain non-coding 5′ and 3′ sequences, such as transcribed, non-translated sequences, splicing and polyadenylation signals.

A polynucleotide encoding a CopB polypeptide of the present invention, including homologs and orthologs from species other than Moraxella catarrhalis is obtained by a process which comprises the steps of screening an appropriate library under stringent hybridization conditions (discussed below) with a labeled probe having a polynucleotide sequence encoding a polypeptide fragment of SEQ ID NO:1, SEQ ID NO:2, SEQ ID NO:3, SEQ ID NO:4 or a fragment thereof; and isolating full-length cDNA and genomic clones containing the polynucleotide sequence. Such hybridization techniques are well known to the skilled artisan. The skilled artisan will appreciate that, in many cases, an isolated cDNA sequence will be incomplete, in that the region coding for the polypeptide is cut short at the 5′ end of the cDNA. This is a consequence of reverse transcriptase, an enzyme with inherently low “processivity” (a measure of the ability of the enzyme to remain attached to the template during the polymerization reaction), failing to complete a DNA copy of the mRNA template during 1st strand cDNA synthesis.

Thus, in certain embodiments, the polypeptide sequence information provided by the present invention (i.e., SEQ ID NOs:1-4) allows for the preparation of relatively short DNA (or RNA) oligonucleotide sequences having the ability to specifically hybridize to gene sequences of the selected polynucleotides disclosed hereinafter. The term “oligonucleotide” as used hereinafter is defined as a molecule comprised of two or more deoxyribonucleotides or ribonucleotides, usually more than three (3), and typically more than ten (10) and up to one hundred (100) or more (although preferably between twenty and thirty). The exact size will depend on many factors, which in turn depends on the ultimate function or use of the oligonucleotide. Thus, in particular embodiments of the invention, nucleic acid probes of an appropriate length are prepared based on a consideration of a selected nucleotide sequence. The ability of such nucleic acid probes to specifically hybridize to a polynucleotide encoding a CopB polypeptide lends them particular utility in a variety of embodiments. Most importantly, the probes are used in a variety of assays for detecting the presence of complementary sequences in a given sample.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a CopB polynucleotide that encodes a CopB polypeptide from prokaryotic cells using polymerase chain reaction (PCR) technology.

In certain embodiments, it is advantageous to employ a polynucleotide of the present invention in combination with an appropriate label for detecting hybrid formation. A wide variety of appropriate labels are known in the art, including radioactive, enzymatic or other ligands, such as avidin/biotin, which are capable of giving a detectable signal.

Polynucleotides which are identical or sufficiently identical to a nucleotide sequence encoding a polypeptide fragment of SEQ ID NOs:1-4, may be used as hybridization probes for CDNA and genomnic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than Moraxella catarrhalis) that have a high sequence similarity to a polynucleotide encoding a polypeptide fragment of sequence of SEQ ID NOs:1-4. Typically these nucleotide sequences are from at least about 70% identical to at least about 95% identical to that of the reference polynucleotide sequence. The probes or primers will generally comprise at least 15 nucleotides, and may have at least 18, 21, 24, 30, 40, or 50 nucleotides. Particularly preferred probes will have between 15 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (BD Biosciences Clontech, Palo Alto, Calif.) for example, have significantly simplified the search for longer cDNAs.

To provide certain of the advantages in accordance with the present invention, a preferred nucleic acid sequence employed for hybridization studies or assays includes probe molecules that are complementary to at least a 10 to about 18 nucleotides long stretch of a polynucleotide encoding a polypeptide of SEQ ID NO:1-4. A size of at least 10 nucleotides in length helps to ensure that the fragment will be of sufficient length to form a duplex molecule that is both stable and selective. Molecules having complementary sequences over stretches greater than 10 bases in length are generally preferred, though, in order to increase stability and selectivity of the hybrid, and thereby improve the quality and degree of specific hybrid molecules obtained. Such fragments are readily prepared by, for example, directly synthesizing the fragment by chemical means, by application of nucleic acid reproduction technology, such as the PCR technology of (U.S. Pat. No. 4,683,202, incorporated hereinafter by reference) or by excising selected DNA fragments from recombinant plasmids containing appropriate inserts and suitable restriction enzyme sites.

In another aspect, the present invention contemplates an isolated and purified polynucleotide comprising a nucleotide sequence that is identical or complementary to a segment of at least 10 contiguous bases of a polynucleotide encoding a polypeptide of SEQ ID NO:1-4 wherein the polynucleotide hybridizes to a polynucleotide that encodes a CopB polypeptide comprising the CopB epitope domain.

Accordingly, a polynucleotide probe molecule of the invention is used for its ability to selectively form duplex molecules with complementary stretches of the gene. Depending on the application envisioned, one will desire to employ varying conditions of hybridization stringency to achieve varying degree of selectivity of the probe toward the target sequence (see Table 3 below). For applications requiring a high degree of selectivity, one will typically desire to employ relatively stringent conditions to form the hybrids. For some applications, for example, where one desires to prepare mutants employing a mutant primer strand hybridized to an underlying template or where one seeks to isolate a homologous polypeptide coding sequence from other cells, functional equivalents, or the like, less stringent hybridization conditions are typically needed to allow formation of the heteroduplex (see Table 3). Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions are readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described hereinafter. Examples of stringency conditions are shown in Table 3 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 3
Hybridization Stringency Conditions

| | Poly- | Hybrid | Hybridization | Wash |
|---|---|---|---|---|
| Stringency | nucleotide | Length | Temperature | Temperature |
| Condition | Hybrid | (bp)^{I} | and Buffer^{H} | and Buffer^{H} |
| A | DNA:DNA | >50 | 65° C.; 1xSSC -or- | 65° C.; |
| | | | 42° C.; 1xSSC, 50% | 0.3xSSC |
| | | | formamide | |
| B | DNA:DNA | <50 | T_{B}; 1xSSC | T_{B}; 1xSSC |
| C | DNA:RNA | >50 | 67° C.; 1xSSC -or- | 67° C.; |
| | | | 45° C.; 1xSSC, 50% | 0.3xSSC |
| | | | formamide | |
| D | DNA:RNA | <50 | T_{D}; 1xSSC | T_{D}; 1xSSC |
| E | RNA:RNA | >50 | 70° C.; 1xSSC -or- | 70° C.; |
| | | | 50° C.; 1xSSC, 50% | 0.3xSSC |
| | | | formamide | |
| F | RNA:RNA | <50 | T_{F}; 1xSSC | T_{F}; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}; 4xSSC | T_{H}; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}; 4xSSC | T_{J}; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}; 2xSSC | T_{L}; 2xSSC |
| M | DNA:DNA | >50 | 50° C.; 4xSSC -or- | 50° C.; 2xSSC |
| | | | 40° C.; 6xSSC, 50% | |
| | | | formamide | |
| N | DNA:DNA | <50 | T_{N}; 6xSSC | T_{N}; 6xSSC |
| O | DNA:RNA | >50 | 55° C.; 4xSSC -or- | 55° C.; 2xSSC |
| | | | 42° C.; 6xSSC, 50% | |
| | | | formamide | |
| P | DNA:RNA | <50 | T_{P}; 6xSSC | T_{P}; 6xSSC |
| Q | RNA:RNA | >50 | 60° C.; 4xSSC -or- | 60° C.; 2xSSC |
| | | | 45° C.; 6xSSC, 50% | |
| | | | formamide | |
| R | RNA:RNA | <50 | T_{R}; 4xSSC | T_{R}; 4xSSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| # Tₘ(° C.) = 81.5 + 16.6 (log₁₀[Na⁺]) + 0.41(% G + C) − (600/N), where N is the number of bases in the hybrid, and [Na⁺] is the concentration of sodium ions in the hybridization buffer ([Na⁺] for 1xSSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated hereinafter by reference.

### C. RECOMBINANT EXPRESSION VECTORS AND HOST CELLS

In another embodiment, the present invention provides expression vectors comprising polynucleotides that encode Moraxella catarrhalis CopB polypeptides. Preferably, an expression vector of the present invention comprises a polynucleotide that encodes a CopB polypeptide comprising the amino acid sequence of SEQ ID NO:1, SEQ ID NO:2, SEQ ID NO:3, or SEQ ID NO:4. Even more preferably, the expression vectors of the invention comprise polynucleotides operatively linked to an enhancer-promoter. In certain embodiments, the expression vectors of the invention comprise polynucleotides operatively linked to a prokaryotic promoter.

As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Expression of proteins in prokaryotes is most often carried out in E. coli with vectors containing constitutive or inducible promoters directing the expression of either fusion or non-fusion proteins. Fusion vectors add a number of amino acids to a protein encoded therein, to the amino or carboxy terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith and Johnson,1988), pMAL (New England Biolabs, Beverly; Mass.) and pRIT5 (Pharmacia, Piscataway, N.J.) which fuse glutathione S-transferase (GST), maltose E binding protein, or protein A, respectively, to the target recombinant protein.

In one embodiment, the coding sequence of a CopB gene (e.g., a polynucleotide encoding a polypeptide having a CopB epitope domain) is cloned into a pGEX expression vector to create a vector encoding a fusion protein comprising, from the N-terminus to the C-terminus, GST-thrombin cleavage site-CopB polypeptide. The fusion protein is purified by affinity chromatography using glutathione-agarose resin. Recombinant CopB polypeptides unfused to GST can be recovered by cleavage of the fusion protein with thrombin.

Examples of suitable inducible non-fusion E. coli expression vectors include pTrc (Amann et al., 1988) and pET IId (Studier et al., 1990). Target gene expression from the pTrc vector relies on host RNA polymerase transcription from a hybrid trp-lac fusion promoter. Target gene expression from the pET IId vector relies on transcription from a T7 gn1 β-lac fusion promoter mediated by a coexpressed viral RNA polymerase T7 gnl. This viral polymerase is supplied by host strains BL21 (DE3) or HMS I 74(DE3) from a resident prophage harboring a T7 gnl gene under the transcriptional control of the lacUV 5 promoter.

One strategy to maximize recombinant protein expression in E. coil is to express the protein in a host bacteria with an impaired capacity to proteolytically cleave the recombinant protein. Another strategy is to alter the nucleic acid sequence of the nucleic acid to be inserted into an expression vector so that the individual codons for each amino acid are those preferentially utilized in E. coli. Such alteration of nucleic acid sequences of the invention is carried out by standard DNA mutagenesis or synthesis techniques.

A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Means for operatively linking an enhancer-promoter to a coding sequence are well known in the art. As is also well known in the art, the precise orientation and location relative to a coding sequence whose transcription is controlled, is dependent inter alla upon the specific nature of the enhancer-promoter. Thus, a TATA box minimal promoter is typically located from about 25 to about 30 base pairs upstream of a transcription initiation site and an upstream promoter element is typically located from about 100 to about 200 base pairs upstream of a transcription initiation site. In contrast, an enhancer can be located downstream from the initiation site and can be at a considerable distance from that site.

Transfected cells of the present invention are also useful for eliciting antibody production or for immunizing humans and animals against pathogenic agents. Implanted transfected cells can be used to deliver immunizing antigens that result in stimulation of the host's cellular and humoral immune responses. These immune responses can be designed for protection of the host from Moraxella catarrhalis infection (ie., for immunization), to stimulate and augment the disease-fighting capabilities directed against an ongoing infection, or to produce antibodies directed against the antigen produced in vivo by the transfected cells that can be useful for therapeutic or diagnostic purposes. Removable barrier devices can be used to allow a simple means of terminating exposure to the antigen. Alternatively, the use of cells that will ultimately be rejected (xenogeneic or allogeneic transfected cells) can be used to limit exposure to the antigen, since antigen production will cease when the cells have been rejected.

Another aspect of the invention pertains to host cells into which a recombinant expression vector of the invention has been introduced. The terms “host cell”, “genetically engineered host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a CopB polypeptide epitope domain can be expressed in bacterial cells such as E. coli, Moraxella catarrhalis, insect cells (such as Sf9 or Sf21 cells), yeast (such as S. cerevisiae) or mammalian cells (such as Chinese hamster ovary cells (CHO), NIH3T3, PER.C6, NSO or COS cells). Other suitable host cells are known to those skilled in the art.

Vector DNA can be introduced into prokaryotic or eukaryotic cells via conventional transformation, infection or transfection techniques. As used herein, the terms “transformation”, “infection”, and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, infection or electroporation. Suitable methods for transforming, infecting or transfecting host cells can be found in Sambrook, et al. (“Molecular Cloning: A Laboratory Manual” 2nd ed, Cold Spring Harbor Laboratory, Cold Spring Harbor Laboratory Press, Cold Spring Harbor, N.Y., 1989), and other laboratory manuals.

A host cell of the invention, such as a prokaryotic or eukaryotic host cell in culture, can be used to produce (ie., express) CopB polypeptides. Accordingly, the invention further provides methods for producing CopB polypeptides using the host cells of the invention. In one embodiment, the method comprises culturing the host cell of invention (into which a recombinant expression vector encoding a polypeptide has been introduced) in a suitable medium until the CopB polypeptide is produced. In another embodiment, the method further comprises isolating the CopB polypeptide from the medium or the host cell.

A coding sequence of an expression vector is operatively linked to a transcription terminating region. RNA polymerase transcribes an encoding DNA sequence through a site where polyadenylation occurs. Typically, DNA sequences located a few hundred base pairs downstream of the polyadenylation site serve to terminate transcription. Those DNA sequences are referred to herein as transcription-termination regions. Those regions are required for efficient polyadenylation of transcribed messenger RNA (mRNA). Transcription-terminating regions are well known in the art. A preferred transcription-terminating region used in a vector construct of the present invention comprises a polyadenylation signal of SV40 or the protamine gene. The bGH polyadenylation signal is also suitable for use.

An expression vector comprises a polynucleotide that encodes an CopB polypeptide (e.g., a CopB epitope domain). Such a polypeptide is meant to include a sequence of nucleotide bases encoding a CopB polypeptide sufficient in length to distinguish said segment from a polynucleotide segment encoding a non-CopB polypeptide. A polypeptide of the invention can also encode biologically functional polypeptides or CopB polypeptides which have variant amino acid sequences, such as with changes selected based on considerations such as the relative hydropathic score of the amino acids being exchanged. These variant sequences are those isolated from natural sources or induced in the sequences disclosed herein using a mutagenic procedure such as site-directed mutagenesis.

A DNA molecule, gene or polynucleotide of the present invention can be incorporated into a vector by a number of techniques which are well known in the art. For instance, the vector pUC18 has been demonstrated to be of particular value Likewise, the related vectors M13mp18 and M13mp19 can be used in certain embodiments of the invention, in particular, in performing dideoxy sequencing.

An expression vector of the present invention is useful both as a means for preparing quantities of DNA encoding a CopB polypeptide itself, and as a means for preparing the encoded polypeptides. It is contemplated that where CopB polypeptides of the invention are made by recombinant means, one can employ either prokaryotic or eukaryotic expression vectors as shuttle systems.

In yet another embodiment, the present invention provides recombinant host cells transformed, infected or transfected with polynucleotides that encode CopB polypeptides. Preferably, the recombinant host cells of the present invention are transfected with a polynucleotide encoding a CopB polypeptide epitope domain of SEQ ID NO:1, SEQ ID NO:2, SEQ ID NO:3 or SEQ ID NO:4. Means of transforming or transfecting cells with exogenous polynucleotide such as DNA molecules are well known in the art and include techniques such as calcium-phosphate- or DEAE-dextran-mediated transfection, protoplast fusion, electroporation, liposome mediated transfection, direct microinjection and adenovirus infection (Sambrook, Fritsch and Maniatis, 1989).

The most widely used method is transfection mediated by either calcium phosphate or DEAE-dextran. Although the mechanism remains obscure, it is believed that the transfected DNA enters the cytoplasm of the cell by endocytosis and is transported to the nucleus. Depending on the cell type, up to 90% of a population of cultured cells can be transfected at any one time. Because of its high efficiency, transfection mediated by calcium phosphate or DEAE-dextran is the method of choice for experiments that require transient expression of the foreign DNA in large numbers of cells. Calcium phosphate-mediated transfection is also used to establish cell lines that integrate copies of the foreign DNA, which are usually arranged in head-to-tail tandem arrays into the host cell genome.

In the protoplast fusion method, protoplasts derived from bacteria carrying high numbers of copies of a plasmid of interest are mixed directly with cultured mammalian cells. After fusion of the cell membranes (usually with polyethylene glycol), the contents of the bacteria are delivered into the cytoplasm of the mammalian cells and the plasmid DNA is transported to the nucleus. Protoplast fusion is not as efficient as transfection for many of the cell lines that are commonly used for transient expression assays, but it is useful for cell lines in which endocytosis of DNA occurs inefficiently. Protoplast fusion frequently yields multiple copies of the plasmid DNA tandemly integrated into the host chromosome.

The application of brief, high-voltage electric pulses to a variety of mammalian and plant cells leads to the formation of nanometer-sized pores in the plasma membrane. DNA is taken directly into the cell cytoplasm either through these pores or as a consequence of the redistribution of membrane components that accompanies closure of the pores. Electroporation can be extremely efficient and can be used both for transient expression of cloned genes and for establishment of cell lines that carry integrated copies of the gene of interest. Electroporation, in contrast to calcium phosphate-mediated transfection and protoplast fusion, frequently gives rise to cell lines that carry one, or at most a few, integrated copies of the foreign DNA.

Liposome transfection involves encapsulation of DNA and RNA within liposomes, followed by fusion of the liposomes with the cell membrane. The mechanism of how DNA is delivered into the cell is unclear but transfection efficiencies can be as high as 90%.

Direct microinjection of a DNA molecule into nuclei has the advantage of not exposing DNA to cellular compartments such as low-pH endosomes. Microinjection is therefore used primarily as a method to establish lines of cells that carry integrated copies of the DNA of interest.

The use of adenovirus as a vector for cell transfection is well known in the art. Adenovirus vector-mediated cell transfection has been reported for various cells (Stratford-Perricaudet et al. 1992).

In a preferred embodiment the recombinant host cells of the present invention are prokaryotic host cells. Preferably, the recombinant host cells of the invention are bacterial cells of the DH5 α strain of Escherichia coli. In general, prokaryotes are preferred for the initial cloning of DNA sequences and constructing the vectors useful in the invention. For example, E. coli K12 strains can be particularly useful. Other microbial strains which can be used include E. coli B. and E. coliₓ1976 (ATCC No. 31537). These examples are, of course, intended to be illustrative rather than limiting.

Prokaryotes can also be used for expression. The aforementioned strains, as well as E. coli W3110 (ATCC No. 273325), bacilli such as Bacillus subtilis, or other enterobacteriaceae such as Salmonella typhimurium or Serratia marcesans, and various Pseudomonas species can be used.

In general, plasmid vectors containing replicon and control sequences which are derived from species compatible with the host cell are used in connection with these hosts. The vector ordinarily carries a replication site, as well as marking sequences which are capable of providing phenotypic selection in transformed cells. For example, E. coli is transformed using pBR322, a plasmid derived from an E. coli species (Bolivar et al. 1977). pBR322 contains genes for ampicillin and tetracycline resistance and thus provides an easy means for identifying transformed cells. The pBR plasmid, or other microbial plasmid or phage must also contain, or be modified to contain, promoters which can be used by the microbial organism for expression of its own polypeptides.

Those promoters most commonly used in recombinant DNA construction include the β-lactamase (penicillinase) and lactose promoter systems (Chang et al. 1978; Itakura et aL 1977; Goeddel et al. 1979; Goeddel et aL 1980) and a tryptophan (TRP) promoter system (European Application No. EP 0036776; Siebwenlist et aL 1980). While these are the most commonly used, other microbial promoters have been discovered and utilized, and details concerning their nucleotide sequences have been published, enabling a skilled worker to introduce functional promoters into plasmid vectors (Siebwenlist et al. 1980).

Following transfection, the cell is maintained under culture conditions for a period of time sufficient for expression of CopB polypeptides. Culture conditions are well known in the art and include ionic composition and concentration, temperature, pH and the like. Typically, transfected cells are maintained under culture conditions in a culture medium. Suitable medium for various cell types are well known in the art. In a preferred embodiment, temperature is from about 20° C. to about 50° C., more preferably from about 30° C. to about 40° C. and, even more preferably about 37° C.

The pH is preferably from about a value of 6.0 to a value of about 8.0, more preferably from about a value of about 6.8 to a value of about 7.8 and, most preferably about 7.4. Osmolality is preferably from about 200 milliosmols per liter (mosm/L) to about 400 mosm/l and, more preferably from about 290 mosm/L to about 310 mosm/L. Other biological conditions needed for transfection and expression of an encoded polypeptide are well known in the art.

Transfected cells are maintained for a period of time sufficient for expression of CopB polypeptides. A suitable time depends inter alia upon the cell type used and is readily determinable by a skilled artisan. Typically, maintenance time is from about 2 to about 14 days.

Recombinant CopB polypeptides are recovered or collected either from the transfected cells or the medium in which those cells are cultured. Recovery comprises isolating and purifying the CopB polypeptides. Isolation and purification techniques for polypeptides are well known in the art and include such procedures as precipitation, filtration, chromatography, electrophoresis and the like.

### D. ANTIBODIES IMMUNOREACTIVE WITH CopB Epitopes

In still another embodiment, the present invention provides antibodies immunoreactive with CopB epitope domains. In one embodiment the antibodies of the invention are monoclonal antibodies. In another embodiment the antibodies of the invention are polyclonal antibodies. Additionally, the CopB polypeptides are CopB polypeptide fragments comprising an epitope domain comprise the amino acid sequence of SEQ ID NO:1, SEQ ID NO:2, SEQ ID NO:3, or SEQ ID NO:4. Means for preparing and characterizing antibodies are well known in the art (See, e.g., Antibodies “A Laboratory Manual”, E. Harlow and D. Lane, Cold Spring Harbor Laboratory, 1988).

Briefly, a polyclonal antibody is prepared by immunizing an animal with an immunogen comprising a polypeptide or polynucleotide of the present invention, and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

As is well known in the art, a given polypeptide or polynucleotide may vary in its immunogenicity. It is often necessary therefore to couple the immunogen (e.g., a polypeptide or polynucleotide) of the present invention with a carrier. Exemplary and preferred carriers are CRM₁₉₇, keyhole limpet hemocyanin (KLH) and bovine serum albumin (BSA). Other albumins such as ovalbumin, mouse serum albumin or rabbit serum albumin can also be used as carriers.

Means for conjugating a polypeptide or a polynucleotide to a carrier protein are well known in the art and include glutaraldehyde, m-maleimidobencoyl-N-hydroxysuccinimide ester, carbodiimide and bis-biazotized benzidine.

The amount of immunogen used for the production of polyclonal antibodies varies inter alia, upon the nature of the immunogen as well as the animal used for immunization. A variety of routes can be used to administer the immunogen (subcutaneous, intramuscular, intradermal, intravenous and intraperitoneal). The production of polyclonal antibodies is monitored by sampling blood of the immunized animal at various points following immunization. When a desired level of immunogenicity is obtained, the immunized animal can be bled and the serum isolated and stored.

In another aspect, the present invention contemplates a process of producing an antibody immunoreactive with a CopB polypeptide comprising the steps of: (a) transfecting recombinant host cells with a polynucleotide that encodes a CopB polypeptide; (b) culturing the host cells under conditions sufficient for expression of the polypeptide; (c) recovering the polypeptides; and (d) preparing the antibodies to the polypeptides. Preferably, the host cell is transfected with the polynucleotide encoding a CopB epitope domain of SEQ ID NO:1, SEQ ID NO:2, SEQ ID NO:3 or SEQ ID NO:4. Even more preferably, the invention provides antibodies prepared according to the process described above.

A monoclonal antibody of the present invention is readily prepared through use of well-known techniques such as those exemplified in U.S. Pat. No. 4,196,265, hereinafter incorporated by reference. Typically, the technique involves first immunizing a suitable animal with a selected antigen (e.g., a polypeptide or polynucleotide of the present invention) in a manner sufficient to provide an immune response. Rodents, such as mice and rats, are preferred animals. Spleen cells from the immunized animal are then fused with cells of an immortal myeloma cell. Where the immunized animal is a mouse, a preferred myeloma cell is a murine NS-1 myeloma cell.

The fused spleen/myeloma cells are cultured in a selective medium to select fused spleen/myeloma cells from the parental cells. Fused cells are separated from the mixture of non-fused parental cells, e.g., by the addition of agents that block the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides. Where azaserine is used, the media is supplemented with hypoxanthine.

This culturing provides a population of hybridomas from which specific hybridomas are selected. Typically, selection of hybridomas is performed by culturing the cells by single-clone dilution in microtiter plates, followed by testing the individual clonal supernatants for reactivity with an antigen-polypeptide. The selected clones are then propagated indefinitely to provide the monoclonal antibody.

By use of a monoclonal antibody of the present invention, specific polypeptides and polynucleotide of the invention are identified as antigens. Once identified, those polypeptides and polynucleotides are isolated and purified by techniques such as antibody-affinity chromatography. In antibody-affinity chromatography, a monoclonal antibody is bound to a solid substrate and exposed to a solution containing the desired antigen. The antigen is removed from the solution through an immunospecific reaction with the bound antibody. The polypeptide or polynucleotide is then easily removed from the substrate and purified.

Additionally, examples of methods and reagents particularly amenable for use in generating and screening an antibody display library can be found in, for example, U.S. Pat. No. 5,223,409; International Application WO 92/18619; International Application WO 91/17271; International Application WO 92/20791; International Application WO 92/15679; International Application WO 93/01288; International Application WO 92/01047; International Application WO 92/09690; International Application WO 90/02809.

Additionally, recombinant anti-CopB antibodies, such as chimeric and humanized monoclonal antibodies, comprising both human and non-human fragments, which can be made using standard recombinant DNA techniques, are within the scope of the invention. Such chimeric and humanized monoclonal antibodies can be produced by recombinant DNA techniques known in the art, for example using methods described in International Application PCT/US86/02269; International Application EP 184,187; International Application EP 171,496; International Application EP 173,494; International Application WO 86/01533; U.S. Pat. No. 4,816,567; and International Application EP 125,023.

An anti-CopB antibody (e.g., monoclonal antibody) is used to isolate CopB polypeptides by standard techniques, such as affinity chromatography or immunoprecipitation. An anti-CopB antibody facilitates the purification of CopB polypeptide from cells and recombinantly produced CopB polypeptides expressed in host cells. Moreover, an anti-CopB antibody is used to detect CopB polypeptide (e.g., in a cellular lysate or cell supernatant) in order to evaluate the abundance of the CopB polypeptide. Anti-CopB antibodies may be used diagnostically to monitor protein levels in tissue as part of a clinical testing procedure, e.g., to, for example, determine the efficacy of a given adjuvant regimen. Detection is facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and acquorin, and examples of suitable radioactive material include ¹²⁵I, ¹³¹I, ¹⁵S or ³H.

### E. IMMUNOGENIC AND PHARMACEUTICAL COMPOSITIONS

In certain preferred embodiments, the present invention provides CopB immunogenic and pharmaceutical compositions comprising CopB polypeptide fragments (ie., CopB epitope domain immunogens) and physiologically acceptable carriers. More preferably, the immunogenic and pharmaceutical compositions comprise a CopB polypeptide fragment having a an amino acid sequence of SEQ ID NO:1, SEQ ID NO:2, SEQ ID NO:3 or SEQ ID NO:4. In a preferred embodiment, an immunogenic composition comprises at least three epitope domains comprising the amino acid sequences of SEQ ID NO:1, SEQ ID NO:2 and SEQ ID NO:3 or SEQ ID NO:4. In other embodiments, the pharmaceutical compositions of the invention comprise polynucleotides that encode CopB polypeptide epitope domains, and physiologically acceptable carriers.

The CopB epitope fragments are incorporated into pharmaceutical and immunogenic compositions suitable for administration to a mammalian subject, e.g., a human. Such compositions typically comprise the “active” composition and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical or immunogenic composition of the invention is formulated to be compatible with its intended route of administration. Examples of routes of administration include parenteral (e.g., intravenous, intradermal, subcutaneous, intramuscular, intraperitoneal), mucosal (e.g., oral, rectal, intranasal, buccal, vaginal, respiratory) and transdermal (topical). Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions is brought about by including in the composition an agent which delays absorption, for example, aluminum monostearate and gelatin.

Sterile injectable solutions are prepared by incorporating the active compound (e.g., a CopB epitope domain) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration can also be by mucosal or transdermal means. For mucosal or transdermal administration, penetrants appropriate to the barrier to be permeated are used in the formulation. Such penetrants are generally known in the art, and include, for example, for mucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds are also prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials are also obtained commercially from Alza Corporation and Nova Pharmaceuticals, Inc. Liposomal suspensions (including liposomes targeted to infected cells with monoclonal antibodies to viral antigens) are also used as pharmaceutically acceptable carriers. These are prepared according to methods known to those skilled in the art, for example, as described in U.S. Pat. No. 4,522,811 which is incorporated hereinafter by reference.

It is especially advantageous to formulate oral or parenteral compositions in dosage unit form for ease of administration and uniformity of dosage. Dosage unit form as used hereinafter refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In preferred embodiments, combination immunogenic compositions are provided by including three or more of the CopB polypeptide fragments of the invention (e.g., epitope domains of SEQ ID Nos:1-4). Multivalent immunogenic compositions directed against various bacteria responsible for causing otitis media comprise three or more of the CopB polypeptides of this invention together with one or more other known Moraxella catarrhalis polypeptides, including, but not limited to, the UspA1, UspA2, Bl, C/D, E and 74 kDa proteins, and/or one or more known nontypable Haemophilus influenzae polypeptides, including, but not limited to, the P2, P4, P5, P6 and PCP proteins, and/or one or more known Streptococcus pneumoniae polypeptides and polysaccharide-protein conjugates, including, but not limited to, the currently available 23-valent pneumococcal capsular polysaccharide vaccine and the 7-valent pneumococcal polysaccharide-protein conjugate vaccine. One particularly preferred multivalent immunogenic composition comprises three or more of the polypeptides of this invention together with the P4, P6 and UspA2 polypeptides.

A pharmaceutically acceptable vehicle is understood to designate a compound or a combination of compounds entering into a pharmaceutical or immunogenic composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

An “adjuvant” is a substance that serves to enhance the immunogenicity of an immunogen. Thus, adjuvants are often given to boost the immune response and are well known to the skilled artisan. Examples of adjuvants contemplated in the present invention include, but are not limited to, aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Mass.), described in U.S. Pat. No. 5,057,540, a pertussis toxin (PT), or an E. coli heat-labile toxin (LT), particularly LT-K63, LT-R72, CT-Si09, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, preferably a histidine, in accordance with published International Patent Application number WO 00/18434). Various cytokines and lymphokines are suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine lnterleukin-12 (IL-12) is another adjuvant which is described in U.S. Pat. No. 5,723,127. Other cytokines or lymphokines have been shown to have immune modulating activity, including, but not limited to, the interleukins 1-alpha, 1-beta, 2, 4, 5,6, 7, 8, 10, 13, 14, 15, 16, 17 and 18, the interferons-alpha, beta and gamma, granulocyte colony stimulating factor, and the tumor necrosis factors alpha and beta, and are suitable for use as adjuvants.

A composition of the present invention is typically administered parenterally in dosage unit formulations containing standard, well-known nontoxic physiologically acceptable carriers, adjuvants, and vehicles as desired. The term parenteral as used hereinafter includes intravenous, subcutaneous, intradermal, intramuscular, intraarterial injection, or infusion techniques.

Injectable preparations, for example sterile injectable aqueous or oleaginous suspensions, are formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol.

Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid find use in the preparation of injectables.

Preferred carriers include neutral saline solutions buffered with phosphate, lactate, Tris, and the like. When administering viral vectors, the vector is purified sufficiently to render it essentially free of undesirable contaminants, such as defective interfering adenovirus particles or endotoxins and other pyrogens, so that it does not cause any untoward reactions in the individual receiving the vector construct. A preferred means of purifying the vector involves the use of buoyant density gradients, such as cesium chloride gradient centrifugation.

A carrier can also be a liposome. Means for using liposomes as delivery vehicles are well known in the art.

In particular embodiments, an immunogenic composition of this invention comprises a polynucleotide sequence of this invention operatively associated with a regulatory sequence that controls gene expression. The polynucleotide sequence of interest is engineered into an expression vector, such as a plasmid, under the control of regulatory elements which will promote expression of the DNA, that is, promoter and/or enhancer elements. In a preferred embodiment, the human cytomegalovirus immediate-early promoter/enhancer is used (U.S. Pat. No. 5,168,062). The promoter may be cell-specific and permit substantial transcription of the polynucleotide only in predetermined cells.

The polynucleotide is introduced directly into the host either as “naked” DNA (U.S. Pat. No. 5,580,859) or formulated in compositions with agents which facilitate immunization, such as bupivicaine and other local anesthetics (U.S. Pat. No. 5,593,972) and cationic polyamines (U.S. Pat. No. 6,127,170).

In this polynucleotide immunization procedure, the polypeptides of the invention are expressed on a transient basis in vivo; no genetic material is inserted or integrated into the chromosomes of the host. This procedure is to be distinguished from gene therapy, where the goal is to insert or integrate the genetic material of interest into the chromosome. An assay is used to confirm that the polynucleotides administered by immunization do not give rise to a transformed phenotype in the host (U.S. Pat. No. 6,168,918).

All patents and publications cited herein are hereby incorporated by reference.

### F. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Materials and Methods

Moraxelia catarrhalis isolates and monoclonal antibody. The M. catarrhalis isolates O35E, TTA24 and O46E were provided by E. Hansen (University of Texas, Dallas, Tex.). The M. catarrhalis isolates B46 and J261 were provided by G. Doern (University of Iowa, Iowa City, Iowa). The M. catarrhalis isolate 4608.1 was provided by T. Murphy (University of Buffalo, Buffalo, N.Y.). All other isolates were provided by D. Hardy (University of Rochester, Rochester, N.Y.). The bacteria were stored at −70° C. in Mueller-Hinton broth (Difco Laboratories, Detroit, Mich.) containing 40% glycerol and routinely passaged on Mueller-Hinton agar incubated at 37° C. with 5% CO₂. The MAb 10F3 specific for the CopB from isolate O35E was prepared as an ammonium sulfate concentrate of the culture supernatant from a hybridoma clone provided by E. Hansen.

Expression of the CopB Proteins in E. coli. The gene encoding CopB was cloned from isolate O35E into the pET30a T7 expression plasmid (Novagen, Madison Wis.) without the CopB leader sequence and the resulting plasmid was designated pLP141. Similarly, plasmid pLP131 was constructed using the copB gene PCR amplified from isolate TTA24, and cloned into pET28a T7 expression system (Novagen, Madison Wis.). Both plasmids were used to transform the E. coli expression host strain BL21(DE3). For bacterial growth, the transformed E. coli cultures were incubated at 37° C. in Luria-Bertani (LB) media supplemented with 30 μg/ml kanamycin (LB-Kan). To prepare cells for isolation of the recombinant CopB (rCopB) proteins, frozen cells were revived on LB-Kan agar, and then grown overnight in LB-Kan broth. Fresh LB-Kan broth was inoculated with the overnight culture to a final A₆₀₀ of 0.05 and incubated with shaking until an A₆₀₀ of about 1.5 was reached. This culture was induced with 1 mM isopropyl-1-thio-β-D-galactopyranoside (IPTG). Ten minutes later, rifampicin was added to a final concentration of 0.15 mg/ml. Three hours after induction, the bacteria were harvested by low speed centrifugation.

Isolation of the rCopB proteins. The harvested bacterial cells were re-suspended in phosphate buffered saline (PBS) containing 1% Triton X-100 and passed through a French Press twice (Aminco FA-078, SLM Instruments Inc., Urbana, Ill.). The cell lysate was centrifuged at 36,000×g for 20 minutes. The pellet, consisting mainly of rCopB inclusion bodies, was extracted with 6 M urea and 0.5 M sodium chloride in 0.1 M Tris at pH 8.0 overnight at 4° C. with gentle rocking. After centrifugation at 36,000×g for 20 minutes, the soluble rCopB remained in the supernatant and was passed through a size exclusion column (Sephadex G-25, Amersham Biosciences, Piscataway, N.J.) equilibrated with 0.5 M sodium chloride and 1% Triton X-100 in 0.1 M Tris at pH 8. The eluant containing the rCopB was centrifuged to remove particulate material. This soluble rCopB migrated as a band of about 80 kDa in 1% sodium dodecyl sulfate-polyacrylamide gel (12% acrylamide) electrophoresis (SDS-PAGE). The purity of the protein was 90% based on the scanning of a Coomassie stained gel.

Synthesis and coniugation of the CopB Deptides. The CopB peptides were synthesized on the Gilson AMS 422 Multiple Peptide Synthesizer (Gilson Medical Electronics, Inc., Middletown, Wis.) using Fmoc, HOBt, PyBOP double couple chemistry and purified on a reverse phase HPLC (Rainin Instrument, Inc., Woburn, Mass.). These peptides were eighteen amino acids in length, including the reported eight amino acid core epitope and the five flanking residues from each side (See, Table 1). The 035E peptide had a sequence of QAELDNKYAGKGYKLGSK (SEQ ID NO:5); the TTA25 peptide, QAELDDKYAGKGYKLGSK (SEQ ID NO:6); and the 430:345 peptide, LAELNKDYPGQGYKLGKK (SEQ ID NO:7). A cysteine was added to the N-terminus of each peptide in order to conjugate the CopB peptide to the genetically detoxified diphtheria toxin CRM₁₉₇.

To make the peptide conjugate, CRM₁₉₇ was first bromoacetylated with the heterobifunctional cross-linking reagent N-succinimidyl bromoacetate at a 1:1 weight-to-weight ratio. This reaction led to the modification of nineteen lysine residues per CRM₁₉₇ molecule as determined by matrix-assisted laser-desorption/ionization time-of-flight mass spectrometry. The peptide was dissolved in water and immediately added to the bromoacetylated CRM₁₉₇ at a 1:1 weight-to-weight ratio. The mixture was incubated overnight with gentle rocking, then dialyzed against PBS to remove free peptides. The extent of conjugation was assessed by amino acid analysis. Upon acid hydrolysis, one S-carboxymethylcysteine was liberated per peptide thioether linkage to the CRM₁₉₇. In SDS-PAGE, the peptide conjugate migrated with a molecular size greater than that of the non-conjugated CRM₁₉₇.

Generation of immune sera in mice. Both BALB/c and Swiss Webster mice were purchased from Taconic Farms, Germantown, N.Y. To examine the immune response to the rCopB proteins, groups of ten female BALB/c mice, six to eight weeks old, were immunized subcutaneously at weeks 0 and 4 with 20 μg of the recombinant proteins adjuvanted with 50 Ag of 3-O-deacylated monophosphoryl lipid A (MPL™; Corixa Corp., Hamilton, Mont.) and 100 tug of aluminum phosphate. Sera collected at weeks 0, 4 and 6 were pooled for analysis. To prepare polyclonal sera against the CopB peptide conjugates, groups of five female Swiss Webster mice, six to eight week old, were immunized subcutaneously with 10 μg of the peptide conjugate adjuvanted with 20 μg of QS21 at weeks 0, 3 and 6. A pool of week 0 sera and a pool of the week 8 sera were used in the analysis. In addition, mouse polyclonal sera against the recombinant CD protein and the native UspA from the O35E isolate were generated. The week 6 sera from these mice were pooled and used as controls in the assays.

Enzyme-linked immunosorbent assays (ELISA). The total antigen specific immunoglobulin level in the pooled sera was measured with a standard enzyme-linked immunosorbent assay procedure (ELISA). For these assays, Costar® 96-well flat bottom polystyrene medium binding plates (Corning Inc., Corning, N.Y.) were coated overnight with the rCopB protein (in 0.1 M Tris at pH 8 containing 6 M urea and 0.5 M sodium chloride) diluted to 2 μg/ml in a pH 9.6 carbonate buffer. The protein specific antibodies were detected with alkaline phosphatase conjugated goat anti-mouse antibodies (BioSource, Camarilla, Calif.). The antibody titers were expressed as the reciprocal of the serum dilution with an absorbance of 0.1 extrapolated from a linear plot of the logarithm of the absorbance versus the logarithm of the serum sample dilution. In all cases, the week 0 sera exhibited an antibody titer of less than 50. The whole cell titers of the pooled sera toward the various isolates of M. catarrhalis were measured as described by Chen et al, 1996. The week 0 sera displayed a whole cell titer of less than 50 for all the isolates tested.

Sequencing of the copb gene. The PCR primer pairs used to clone the copB gene from isolate 430:345 were oligonucleotides 5′-GGATCCGCCACCATGGCTMTMXIGTTTCAATTATTACCG (SEQ ID NO:8) and 5′-GMTTCCCCATAAAAAGMCACCC (SEQ ID NO:9); from isolates 111:210 and 318:086, oligonucleotides 5′-CATATGGCTGTTAGCCAGCCTAAGG (SEQ ID NO:10) and 5′-CACCAAGCTCTACACTGG (SEQ ID NO:11); and from isolates 218:038 and 417:082, oligonucleotides 5′-CTGACATTGGCGGTGAGT (SEQ ID NO:12) and 5′-GTGCTTGAGTGTTCAGT (SEQ ID NO:13). The oligonucleotides were synthesized on a PerSeptive Biosystems oligonucleotide synthesizer (Applied Biosystems, Foster City, Calif.) using β-cyanoethylphosphoramidite chemistry. The copB gene was amplified by PCR from bacterial cells with the appropriate oligonucleotides in ReddyMix PCR Master Mix (ABgene® House, Surrey, U.K.) and the PCR products were cloned into the pCR®2.1-TOPO® vector (Invitrogen Co., Carlsbad, Calif.). DNA sequencing was carried out using BigDye terminator chemistry with the specifically designed primers on an Applied Biosystems 377 automated DNA sequencer (Applied Biosysytems, Foster City, Calif.). Sequencher™ 4.0.5 software (Gene Codes, Corp., Ann Arbor, Mich.) was used to analyze the DNA sequences and the LASERGENE software (DNASTAR, Inc., Madison, Wis.) the protein sequences.

### Example 2

### Immunogenicity of the 035E Recombinent CopB and TTA24 Recombinant CopB Proteins

Both the O35E rCopB and TTA24 rCopB proteins elicited good primary immune responses in mice that were boosted upon a second immunization. The antibodies to the 035E rCopB and the antibodies to the TTA24 rCopB were cross-reactive, i.e., the polyclonal sera raised by either rCopB had high antibody titers toward the homologous as well as the heterologous forms of the protein (See, Table 4).

TABLE 4
Protein specific antibody titers of sera from mice immunized with
035E rCopB, TTA24 rCopB and a mixture of the two proteins

| | Antibody titer^{a} elicited by | | |
|---|---|---|---|
| | 035E | TTA24 | rCopB |
| Detection antigen | rCopB | rCopB | mixture |
| 035E rCopB | 659,950 | 109,290 | 272,075 |
| TTA24 rCopB | 194,086 | 185,260 | 365,430 |

^{a}Antibody titers for the week 6 sera pooled from the ten mice.

High antibody titers toward the two rCopB proteins were also seen for the sera obtained from mice immunized with a mixture of the two recombinant proteins. These results were consistent with the fact that the 035E and TTA24 CopB proteins share greater than 98% sequence identity (Sethi, 1997; Helminen et al., 1993). Considerably less cross-reactivity was detected in the whole cell ELISA assay (Table 5).

TABLE 5
Moraxella catarrhalis whole cell titers of sera from mice immunized with
035E rCopB, TTA24 rCopB and a mixture of the two proteins

| | Reactivity | Whole cell titer^{a} elicited by | | |
|---|---|---|---|---|
| Detection | with | 035E | TTA24 | rCopB |
| isolate | Mab10F3 | rCopB | rCopB | mixture |
| 035E | + | 47,416 | 1,162 | 28,284 |
| 1230:359 | + | 51,855 | 784 | 19,946 |
| 216:96 | + | 41,726 | 639 | 30,609 |
| TTA24 | − | 930 | 4,755 | 42,525 |
| 430:345 | − | 1,772 | 1,038 | 3,301 |
| 111:210 | − | 1,175 | 5,333 | 55,561 |

^{a}Whole cell titers of the week 6 sera pooled from the ten mice.

The anti-035E rCopB serum reacted preferentially with the whole cells of 10F3 positive isolates, and the anti-TTA24 rCopB serum with the whole cells of 10F3 negative isolates, except 430:345. It was observed that the whole cell titers of the anti-TTA24 rCopB serum toward the 10F3 negative isolates were much lower compared to those of the anti-035E rCopB serum toward the 10F3 positive isolates. Nevertheless, the anti-serum raised by a mixture of the two recombinant proteins exhibited comparable whole cell titers to both 10F3 positive and 10F3 negative isolates with the exception of isolate 430:345.

Also examined was the reactivity of the anti-sera in western blots toward M. catarrhalis cell lysates (data not shown). Of the eight tested isolates, the CopB protein (i.e., the band migrating at 80 kDa in SDS-PAGE) of the four 10F3 positive isolates reacted intensely with the polyclonal serum against 035E rCopB, but poorly with the polyclonal serum against TTA24 rCopB. On the other hand, the CopB protein of three of the four 10F3 negative isolates reacted intensely with the polyclonal serum against TTA24 rCopB but poorly with the polyclonal serum against 035E rCopB. Again, it was observed that the CopB of the 10F3 negative isolate 430:345 was an exception. It reacted weakly with both the anti-serum against 035E rCopB and the anti-serum against TTA24 rCopB. The specificity of the polyclonal sera elicited by the two rCopB proteins observed in western blots and in the whole cell ELISA assay strongly suggested that the epitope defined by MAb 10F3 was a single immuno-dominant domain on the surface of the bacterium.

### Example 3

### Detection of a Novel CopB Serogroup

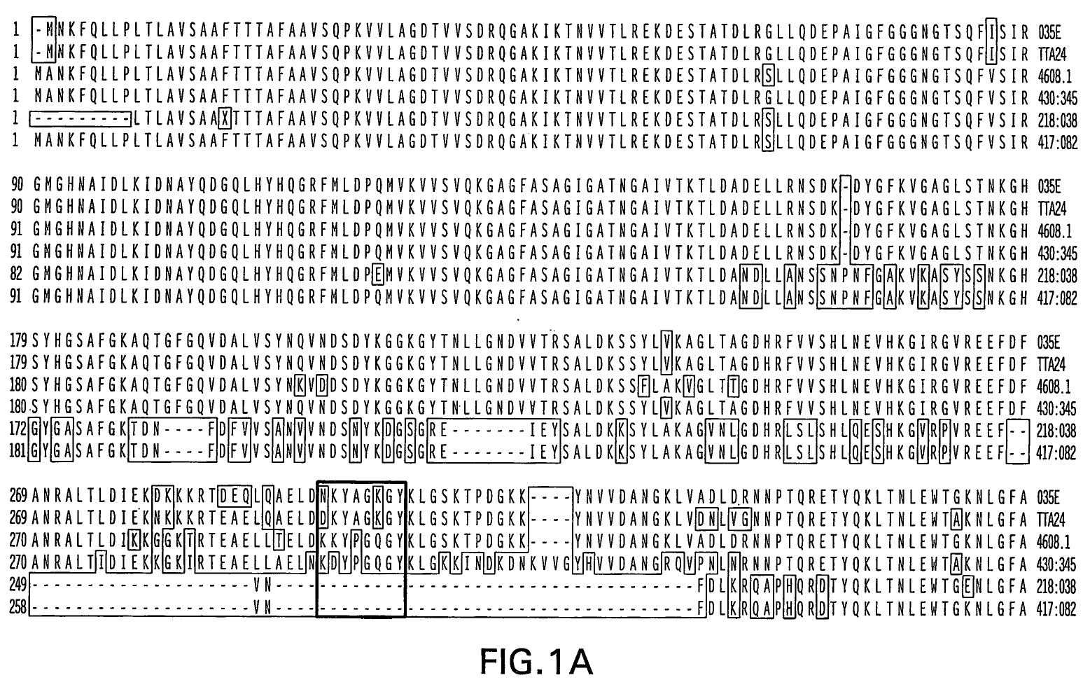

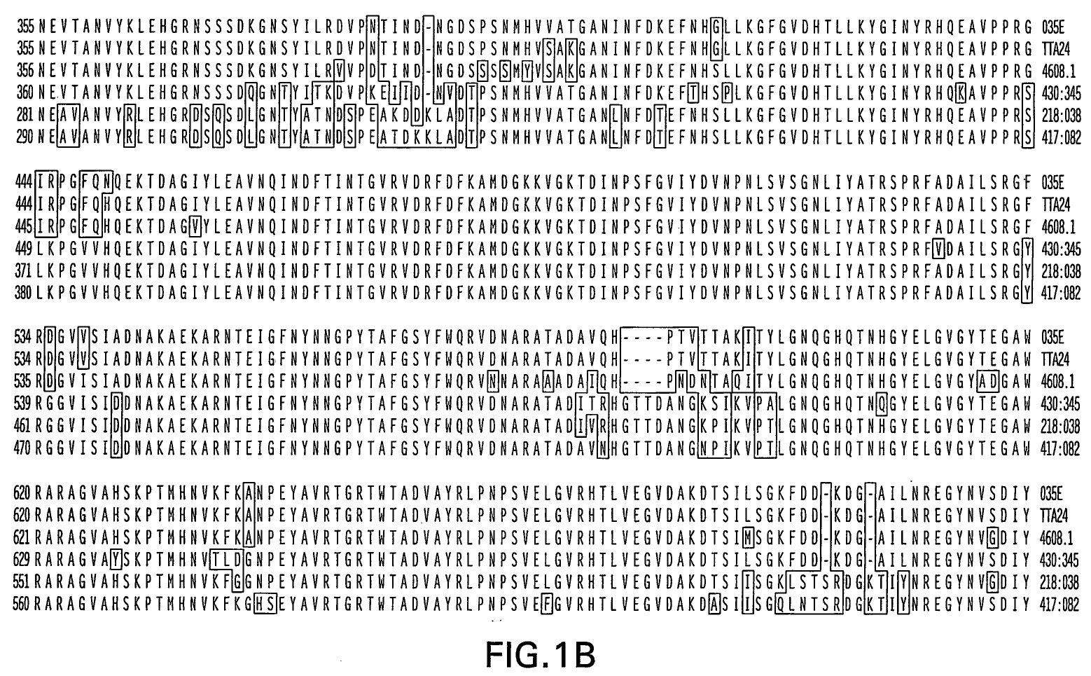

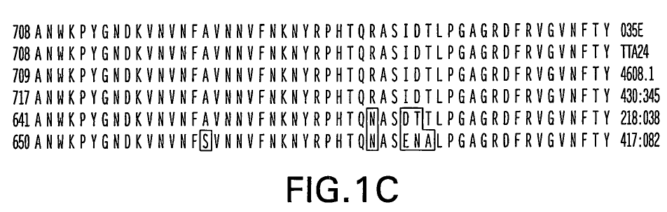

It was of particular interest that the polyclonal sera against the 035E and TTA24 rCopB proteins had relatively low reactivities toward the CopB of the 10F3 negative isolate 430:345 (See Table 5). Thus, it was contemplated that the 430:345 isolate might represent a new CopB serogroup. To test this hypothesis, the copB gene from isolate 430:345 was partially sequenced in the region corresponding to the 10F3 epitope and a four amino acid difference was detected from that of the O35E isolate (FIG. 1). This finding is consistent with the single immuno-dominant epitope hypothesis, and explains why the CopB protein of the 430:345 isolate was immunologically different from the CopB proteins of the 035E or TTA24 isolates.

That the epitope sequence found in the CopB protein of the 430:345 isolate is also present in the published sequences of the CopB of isolates 046E and 56 (Sethi, 1997; Aebi et al., 1998) suggests that these three isolates belong to the same serological group (See Table 1).

### Example 4

### Serology of CopB Characterized by Western Blot Analysis

To estimate the prevalence of this new serogroup among the 10F3 negative isolates, more than a hundred isolates from patients infected with Moraxella catarrhalis were screened by dot blot analysis for reactivity with MAb 10F3. The eighteen isolates that did not react with 10F3, along with four that did react, were selected for further study. Total lysates of the selected isolates were examined by western blot for their reactivity with MAb 10F3 and with the polyclonal sera against the same epitope region from the three CopB serogroups. For this, three peptides, eighteen amino acids in length, were synthesized. Each carried the respective eight amino acid core epitope and five flanking residues on each side as found in the peptide sequence of isolates 035E, TTA24 and 430:345. The peptides were conjugated to CRM₁₉₇ via their N-termini through a cysteine residue, and the peptide conjugates were administered to mice to generate anti-sera. For ease of discussion, the resulting sera are referred to as the anti-035E peptide, anti-TTA24 peptide and anti-430:345 peptide sera. Using these epitope specific antibody reagents, along with the sera prepared against the two rCopB proteins and DNA sequencing, led to the discovery of four serogroups: CopB-I, CopB-II, CopB-III and CopB-IV.

All twenty-two isolates were examined by western blot analysis (data not shown). The specificity of MAb 10F3 was clearly demonstrated by its reactivity toward the CopB protein of isolates 035E, 1230:359, 110:070, 115:142 and B46 (serogroup CopB-I). The anti-TTA24 peptide serum was almost as selective; it reacted most strongly with the CopB protein of three 10F3 negative isolates, TTA24, 111:210 and 113:136 (serogroup CopB-II). The anti-035E peptide serum was much less specific, recognizing the CopB protein of all the isolates except that of 430:345.

In contrast, the reactivity pattern for the anti-430:345 peptide serum was more complex. While displaying weak reactivity toward a number of proteins of various molecular weights in most of the isolates, the anti-430:345 peptide serum only reacted strongly with the CopB protein of 430:345 and 046E (data not shown). This observation further demonstrates that isolates 430:345 and 046E represent a new serological group (serogroup CopB-III).

### Example 5

### Further Characterization of CopB Serogroups by Whole Cell ELISA

The finding of four CopB serogroups prompted further examination of the epitopes of the protein that might be exposed on the bacterial surface. To do so, the whole cell ELISA assay was used to analyze the sera against the two recombinant CopB proteins and the three CopB peptide conjugates. Based on the whole cell titers (Table 6), the twenty-two isolates were sorted into the same four CopB serogroups previously identified by western blot analysis. As summarized in Table 7, serogroup CopB-I was defined by the specific reactivity of those isolates with MAb 10F3 and the anti-035E rCopB serum; serogroup CopB-II with the anti-TTA24 rCopB serum and serogroup CopB-III with the anti-430:345 peptide serum. The sera against the O35E and TTA24 peptides exhibited reactivity patterns similar to those seen for the sera against the rCopB proteins from those isolates, but had lower titers (data not shown). The relatively stringent specificity of these polyclonal sera toward the rCopB proteins and the three CopB peptides clearly indicated that each of the serogroups CopB-I, CopB-II and CopB-III, possessed a single immuno-dominant domain on the bacterial surface of their respective isolates.

TABLE 6
M. catarrhalis whole cell titers of MAb 10F3 and mouse polyclonal
sera prepared against 035E rCopB, TTA24 rCopB, and 430:345
CopB peptide

| | | Whole cell titer | | | |
|---|---|---|---|---|---|
| | | | Anti- | Anti- | Anti- |
| | | | O35E | TTA24 | 430:345 |
| | | | rCopB | rCopB | peptide |
| Serogroup | Isolate | 10F3 Mab | serum | serum | serum |
| CopB-I | O35E | 70,928 | 52,717 | 241 | <100 |
| | 125:114 | 52,257 | 74,909 | 695 | 182 |
| | 128:179 | 19,110 | 18,878 | 240 | <100 |
| | 1230:359 | 7,955 | 65,347 | 553 | <100 |
| | 205:211 | 93,054 | 85,959 | 362 | <100 |
| | 110:070 | 89,037 | 207,562 | 300 | 164 |
| | 306:155 | 138,335 | 173,249 | 660 | 107 |
| | 204:206 | 51,458 | 36,294 | 1,683 | <100 |
| | B46 | 53,866 | 46,408 | 345 | <100 |
| CopB-II | TTA24 | <100 | 382 | 3,340 | <100 |
| | 422:318 | <100 | 304 | 4,166 | <100 |
| | 312:171 | <100 | 1,009 | 9,211 | <100 |
| | J261 | <100 | 514 | 11,601 | 150 |
| | 113:136 | <100 | 316 | 2,710 | <100 |
| | 205:333 | <100 | 807 | 6,169 | <100 |
| CopB-III | 430:345 | <100 | 778 | 822 | 45,583 |
| | O46E | <100 | 576 | 681 | 45,186 |
| CopB-IV | 4608.1 | <100 | 860 | 1,173 | <100 |

TABLE 7
CopB serogroups based on the reactivity observed in the whole cell ELISA
assay

| | Whole cell reactivity toward | | | |
|---|---|---|---|---|
| | | Anti-035E | Anti-TTA24 | Anti-430:345 |
| Serogroup | Mab10F3 | rCopB serum | rCopB serum | peptide serum |
| CopB-I | +++ | +++ | − | − |
| CopB II | − | − | ++ | − |
| CopB-III | − | − | − | +++ |

The characteristics of the four CopB serogroups identified, based on the whole cell reactivity of the twenty-two isolates with MAb 10F3, the anti-sera against the 035E and TTA24 rCopB proteins, and the serum against the 430:345 peptide, are summarized in Table 7. The lack of cross-reactivity of the polyclonal antibodies demonstrated that serogroups CopB-I, CopB-II and CopB-IIl were each defined by a single but different immuno-dominant epitope on the bacterial surface.

### Example 6

### Sequencing of the CopB Genes from CopB-III and CopB-IV Isolates

The copB genes of isolates 430:345 and 4608.1 were cloned and completely sequenced. The predicted CopB protein sequences of these isolates were then compared to the published ones. An alignment of representative CopB protein sequences is shown in FIG. 1. The sequence of the predicted protein for isolate 430:345 was similar to that of isolate 046E, with only five residue differences. In contrast, the sequence of the predicted protein for isolate 430:345 was quite different from that of isolates 035E and TTA24. There were seventy-five or more residue differences different from them. Interestingly, the same immuno-dominant epitope sequence KDYPGQGY (SEQ ID NO:3) was found in the CopB protein of isolates 430:345, 046E and 56 (FIG. 1 and Table 1). The presence of the same epitope domain in isolates 430:345 and 046E correlated well with the specific reactivity of the anti-430:345 peptide serum toward these two isolates in the western blot (data not shown) and the whole cell ELISA assay (Table 6). Although not tested, one would expect isolate 56 to bind the antibodies prepared against the 430:345 peptide as well.

Serogroup CopB-IV isolate did not exhibit reactivity in western blots with either the MAb 10F3 or any of the anti-CopB peptide sera (data not shown), nor did it react with the sero-specific antibody reagents in the whole cell ELISA assay (Table 6). Nevertheless, Applicants were able to clone and sequence the copB gene from this isolate. As shown in FIG. 1, the CopB protein predicted from the copB genes of isolate 4608.1 was quite different from those of the 035E, TTA24 and 430:435 isolates. To determine if a copB gene product is made by this isolate, cell lysates of 4608.1 were probed in western blots with the polyclonal sera against 035E and TTA24rCopB. As mentioned earlier, the polyclonal sera against the rCopB proteins contained antibodies to regions other than the surface exposed immuno-dominant epitope (Table 4). It was found that the 4608.1 isolate expressed a CopB protein of the expected size. These results indicate that 4608.1 represent a new serogroup, which has been designated CopB-IV.

### REFERENCES

International Application No. EP 125,023
International Application No. EP 171,496
International Application No. EP 184,187
International Application No. EP 173,494
International Application No. EP 264166
International Application No. PCT/US86/02269
International Application No. WO 86/01533
International Application No. WO 91/17271
International Application No. WO 92/01047
International Application No. WO 92/09690
International Application No. WO 92/15679
International Application No. WO 92/18619
International Application No. WO 92/20791
International Application No. WO 93/01288
International Application No. WO 00/63364
International Application No. WO 94/12650
International Application No. WO 93/13302
International Application No. WO 92/19265
International Application No. WO 00/18434
International Application No. WO 93/13202
International Application No. WO 96/05858
International Application No. WO 98/20734
International Patent No. WO 98/42375
U.S. Pat. No. 4,196,265
U.S. Pat. No. 4,816,567
U.S. Pat. No. 4,873,316
U.S. Pat. No. 4,987,071
U.S. Pat. No. 4,912,094
U.S. Pat. No. 4,837,151
U.S. Pat. No. 4,522,811
U.S. Pat. No. 4,554,101
U.S. Pat. No. 4,683,202
U.S. Pat. No. 4,987,071
U.S. Pat. No. 5,968,502
U.S. Pat. No. 5,223,409
U.S. Pat. No. 5,116,742
U.S. Pat. No. 5,723,127
U.S. Pat. No. 5,643,576
U.S. Pat. No. 5,593,972
U.S. Pat. No. 5,580,859
U.S. Pat. No. 5,168,062
U.S. Pat. No. 5,078,996
U.S. Pat. No. 5,057,540
U.S. Pat. No. 5,116,742
U.S. Pat. No. 5,108,744
U.S. Pat. No. 5,593,972
U.S. Pat. No. 5,601,831
U.S. Pat. No. 5,614,382
U.S. Pat. No. 5,785,973
U.S. Pat. No. 6,207,646
U.S. Pat. No. 6,168,918
U.S. Pat. No. 6,127,170
U.S. Pat. No. 6,113,918
Aebi et al., “Expression of the CopB outer membrane protein by Moraxella catarrhalis is regulated by iron and affects iron acquisition from transferrin and lactoferrin,” Infect Immun, 64(6), 2024-2030, 1996.
Aebi et al., “Mapping of a protective epitope of the CopB outer membrane protein of Moraxella catarrhalis,” Infect Immun, 66(2), 540-548, 1998
Amann et al., Gene 69:301-315, 1988.
Ameen, Sethi, & Murphy, “Mapping of monoclonal antibodies 1F5 and 2.9F to the first variable region of CopB (OMP B2) of Moraxella catarrhalis,” in 98th General Meeting of the American Society for Microbiology, Atlanta, Ga., 1998. 115.
Anonymous, “Consensus”, Pediatr. Infect. Dis. J., 8:594-597, 1989.
Ausubel et al., 1995, Current Protocols in Molecular Biology, eds., John Wiley & Sons, Inc., sections 2.10 and 6.3-6.4
Baldari et al., Embo J. 6:229-234,1987.
Banerji et al., Cell, 33:729-740; 1983.
Barany et al., Int. J. Peptide Protein Res. 30, 705-739, 1987.
Bennett, Haggard, Silva, & Stewart, “Behavior and developmental effects of otitis media with effusion into the teens,” Arch. Dis. Child., 85, 91-95, 2001.
Bootsma, van der Heide, van de Pas, Schouls, & Mooi, “Analysis of Moraxella catarrhalis by DNA typing: evidence for a distinct subpopulation associated with virulence traits,” J. Infect Dis., 181,1376-1387, 2000.
Byrne and Ruddle, PNAS 86:5473- 5477, 1989.
Calame and Eaton, Adv. Immunol. 43:235-275, 1988.
Campes and Tilghman, Genes Dev. 3:537-546, 1989.
Campagnari, Shanks & Dyer, “Growth of Moraxella catarrhalis with human transferrin and lactoferrin: expression of iron-repressible proteins without siderophore production,” Infection and Immunity, 62(11), 4909-4914, 1994.
Carson, Klebba, Newton, & Sparling, “Ferric enterobactin binding and utilization by Neisseria gonorrhoeae,” J. Bacteriol., 181(9), 2895-2901, 1999.
Chen et al., “Evaluation of purified UspA from Moraxella catarrhalis as a vaccine in a murine model after active immunization,” Infect. Immun., 64(6), 1900-1905, 1996.
de Velasco et al., “Synthetic peptides representing T-cell epitopes act as carriers in pneumococcal polysaccharide conjugate vaccines,” Infect. Immun., 63:961-968, 1995.
Edlund et al., Science 230:912-916, 1985.
Frohman et al., Proc. Natl. Acad. Sci. USA 85, 8998-9002, 1988.
Gaultier et al., Nucleic Acids Res. 15:6625-6641, 1987.
Gentz et al., Proc. Natl. Acad. Sci. USA, 86:821-824, 1989.
Gray, Conjugate Vaccines Supplement p 694-697, 1990.
Harlow and Lane, “Antibodies: A Laboratory Manual,” Cold Spring Harbor Laboratory Press, Cold Spring Harbor, N.Y., 1988
Harrington et al., Nature Biotechnology, 19:440-445, 2001.
Haselhoff and Gerlach, Nature 334:585-591, 1988.
Helene et al., Ann. N.Y Acad Sci. 660:27-36, 1992.
Helene, Anticancer Drug Des. 6(6):569-84, 1991.
Helminen et al., “A mutation affecting expression of a major outer membrane protein of Moraxella catarrhalis alters serum resistance and survival in vivo,” J. Infect Dis., 168, 1194-1201, 1993a.
Helminen, Maciver, Latimer, Cope, McCracken, & Hansen, “A major outer membrane protein of Moraxella catarrhalis is a target for antibodies that enhance pulmonary clearance of the pathogen in an animal model,” Infect Immun, 61(5), 2003-2010, 1993b.
Henriksen et al., “Vaccination with protein-conjugated and native type 3 capsular polysaccharide in an ethanol-ted rat model of pneumococcal pneumonia”, Alcohol Clin. Exp. Res., 21:1630-1637, 1997.
Inoue et al., FEBS Lett. 215:327-330, 1987(a).
Inoue et al., Nucleic Acids Res. 15:6131-6148, 1987(b).
Johnson et al., Endoc. Rev., 10:317-331, 1989.
Kaufman et al., EMBO J 6:187-195, 1987.
Kessel and Gruss, Science 249:3 74-3 79, 1990.
Kurj an and Herskowitz, Cell 933-943, 1982.
Kyte and Doolittle, J. Mol. Biol., 157:105-132, 1982.
Lafontaine, Cope, Aebi, Latimer, McCracken, & Hansen, “The UspA1 protein and a second type of UspA2 protein mediate adherence of Moraxella catarrhalis to human epithelial cells in vitro,” Bacteriol., 182(5), 1364-1373, 2000.
Lucklow and Summers, Virology 170:31-39, 1989.
Maher, Bioassays 14(12):807-15, 1992.
Murphy, “Branhamella catarrhalis: epidemiology, surface antigenic structure, and immune response,” Microbiol. Rev., 60, 267-279, 1996.
Pinkert et al. Genes Dev. 1:268-277, 1987.
Queen and Baltimore, Cell 33:741-748, 1983.
Sambrook et al., “Molecular Cloning: A Laboratory Manual” 2nd ed, Cold Spring Harbor Laboratory, Cold Spring Harbor Laboratory Press, Cold Spring Harbor, N.Y., 1989
Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.
Sasaki, Harkness, Loosmore, Chong, & Klein, “High molecular weight major outer membrane protein of Moraxella,” in Patent Aventis, United States, 2001.
Schultz et al., Gene 54:113-123, 1987
Seed, Nature329:840, 1987.
Sethi & Murphy, “Bacterial infection in chronic obstructive pulmonary disease in 2000, a state-of-the-art review.” Clin. Microbiol. Rev., 14(2), 336-363, 2001.
Sethi, Surface, & Murphy, “Antigenic heterogeneity and molecular analysis of CopB of Moraxella (Branhamella) catarrhalis,” Infect Immun, 65(9), 3666-3671, 1997.
Smith and Johnson, Gene 67:31-40, 1988.
Smith et al., Mol. Cell Biol. 3:2156-2165, 1983.
Studier et al. “Gene Expression Technology” Methods in Enzymology 185, 60-89, 1990.
Tucker, Plosila & Tillman, “Moraxella catarrhalis outer membrane protein-106 polypeptide, gene sequence and uses thereof. In United States Patent, Antex Biologics Inc., United States, 2001.
Winoto and Baltimore. EMBO J 8:729-733, 1989.

What is claimed is:

1. An immunogenic composition comprising Moraxella catarrhalis polypeptide fragments, wherein the fragments comprise amino acid sequences of SEQ ID NO:1, SEQ ID NO:2 and SEQ ID NO:3.

2. The immunogenic composition according to claim 1, wherein the fragments are further defined as CopB epitopes.

3. The immunogenic composition according to claim 1, wherein SEQ ID NO:3 is further defined as an amino acid sequence comprised within a CopB polypeptide of Moraxella catarrhalis isolate 430:345, Moraxella catarrhalls isolate 046E or Moraxella catarrhalis isolate 56.

4. The immunogenic composition according to claim 1, wherein the fragments are conjugated to a carrier protein.

5. The immunogenic composition according to claim 1, further comprising one or more adjuvants.

6. The immunogenic composition according to claim 1, wherein the composition, when administered to a mammalian host in an immunogenic amount, protects the host against greater than 75% of Moraxella catarrhalis strains.

7. The immunogenic composition according to claim 1, further comprising additional antigens.

8. An immunogenic composition comprising Moraxella catarrhalis polypeptide fragments, wherein the fragments comprise amino acid sequences of SEQ ID NO:1, SEQ ID NO:2 and SEQ ID NO:4.

9. The immunogenic composition according to claim 8, wherein the fragments are further defined as CopB epitopes.

10. The immunogenic composition according to claim 8, wherein SEQ ID NO:4 is further defined as an amino acid sequence comprised within a CopB polypepude of Moraxella catarrhalis isolate 4608.1.

11. The immunogenic composition according to claim 8, wherein the fragments are conjugated to a carrier protein.

12. The immunogenic composition according to claim 8, further comprising one or more adjuvants.

13. The immunogenic composition according to claim 8, wherein the composition, when administered to a mammalian host in an immunogenic amount, protects the host against greater than 75% of Moraxella catarrhalis strains.

14. The immunogenic composition according to claim 8, further comprising additional antigens.

15. An immunogenic composition comprising Moraxella catarrhalis polypeptide fragments, wherein the fragments comprise amino acid sequences of SEQ ID NO:1, SEQ ID NO:2, SEQ ID NO:3 and SEQ ID NO:4.

16. The immunogenic composition according to claim 15, wherein the fragments are further defined as CopB epitopes.

17. The immunogenic composition according to claim 15, wherein SEQ ID NO:3 is further defined as an amino acid sequence comprised within a CopB polypeptide of Moraxella catarrhalis isolate 430:345, Moraxella catarrhalis isolate 046E or Moraxella catarrhalis isolate 56.

18. The immunogenic composition according to claim 15, wherein SEQ ID NO:4 is further defined as an amino acid sequence comprised within a CopB polypeptide of Moraxella catarrhalis isolate 4608.1.

19. The immunogenic composition according to claim 15, wherein the fragments are conjugated to a carrier protein.

20. The immunogenic composition according to claim 15, further comprising one or more adjuvants.

21. The immunogenic composition according to claim 15, wherein the composition, when administered to a mammalian host in an immunogenic amount, protects the host against greater than 75% of Moraxella catarrhalis strains.

22. The immunogenic composition according to claim 15, further comprising additional antigens.

23. An immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:3.

24. The immunogenic composition according to claim 23, wherein the polypeptides are further defined as CopB polypeptides.

25. The immunogenic composition of claim 23, wherein SEQ ID NO:3 is further defined as an amino acid sequence comprised within a CopB polypeptide of Moraxella catarrhalis isolate 430:345, Moraxella catarrhalis isolate 046E or Moraxella catarrhalls isolate 56.

26. The immunogenic composition according to claim 23, wherein the polypeptides are conjugated to a carrier protein.

27. The immunogenic composition according to claim 23, further comprising one or more adjuvants.

28. The immunogenic composition according to claim 23, wherein the composition, when administered to a mammalian host in an immunogenic amount, protects the host against greater than 75% of Moraxella catarrhalis strains.

29. The immunogenic composition according to claim 23, further comprising additional antigens.

30. An immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:4.

31. The immunogenic composition according to claim 30, wherein the polypeptides are further defined as CopB polypeptides.

32. The immunogenic composition of claim 30, wherein SEQ ID NO:4 is further defined as an amino acid sequence comprised within a CopB polypeptide of Moraxella catarrhalis isolate 4608.1.

33. The immunogenic composition according to claim 30, wherein the polypeptides are conjugated to a carrier protein.

34. The immunogenic composition according to claim 30, further comprising one or more adjuvants.

35. The immunogenic composition according to claim 30, wherein the composition, when administered to a mammalian host in an immunogenic amount, protects the host against greater than 75% of Moraxella catarrhalis strains.

36. The immunogenic composition according to claim 30, further comprising additional antigens.

37. An immunogenic composition comprising at least four Moraxella catarrhalis polypeptides, wherein the first polypeptide comprises at least the amino acid sequence of SEQ ID NO:1, the second polypeptide comprises at least the amino acid sequence of SEQ ID NO:2, the third polypeptide comprises at least the amino acid sequence of SEQ ID NO:3 and the fourth polypeptide comprises at least the amino acid sequence of SEQ ID NO:4.

38. The immunogenic composition according to claim 37, wherein the polypeptides are further defined as CopB polypeptides.

39. The immunogenic composition of claim 37, wherein SEQ ID NO:3 is further defined as an amino acid sequence comprised within a CopB polypeptide of Moraxella catarrhalis isolate 430:345, Moraxella catarrhalis isolate 046E or Moraxella catarrhalis isolate 56.

40. The immunogenic composition of claim 37, wherein SEQ ID NO:4 is further defined as an amino acid-sequence comprised within a CopB polypeptide of Moraxella catarrhalis isolate 4608.1.

41. The immunogenic composition according to claim 37, wherein the polypeptides are conjugated to a carrier protein.

42. The immunogenic composition according to claim 37, further comprising one or more adjuvants.

43. The immunogenic composition according to claim 37, wherein the composition, when administered to a mammalian host in an immunogenic amount, protects the host against greater than 75% of Moraxella catarrhalis strains.

44. The immunogenic composition according to claim 37, further comprising additional antigens.

45. An immunogenic composition comprising a Moraxella catarrhalis polypeptide fragment comprising an amino acid sequence of SEQ ID NO:3.

46. The immunogenic composition according to claim 45, wherein the fragment is further defined as a CopB epitope.

47. The immunogenic composition according to claim 45, wherein SEQ ID NO:3 is further defined as an amino acid sequence comprised within a CopB polypeptide of Moraxella catarrhalis isolate 430:345, Moraxella catarrhalis isolate 046E or Moraxella catarrhalis isolate 56.

48. The immunogenic composition according to claim 45, wherein the fragment is conjugated to a carrier protein.

49. The immunogenic composition according to claim 45, further comprising one or more adjuvants.

50. The immunogenic composition to claim 45, further comprising additional antigens.

51. An immunogenic composition comprising a Moraxella catarrhalis polypeptide fragment comprising an amino acid sequence of SEQ ID NO:4.

52. The immunogenic composition according to claim 51, wherein the fragment is further defined as a CopB epitope.

53. The immunogenic composition of claim 51, wherein SEQ ID NO:4 is further defined as an amino acid sequence comprised within a CopB polypeptide of Moraxella catarrhalis isolate 4608.1.

54. The immunogenic composition according to claim 51, wherein the fragment is conjugated to a carrier protein.

55. The immunogenic composition according to claim 51, further comprising one or more adjuvants.

56. The immunogenic composition according to claim 51, further comprising additional antigens.

57. An immunogenic composition comprising Moraxella catarrhalis polypeptide fragments, wherein the fragments comprise an amino acid sequence of SEQ ID NO:3 and an amino acid sequence of SEQ ID NO:4.

58. The immunogenic composition according to claim 57, wherein the fragments are further defined as CopB epitopes.

59. The immunogenic composition of claim 57, wherein SEQ ID NO:3 is further defined as an amino acid sequence comprised within a CopB polypeptide of Moraxella catarrhalis isolate 430:345, Moraxella catarrhalis isolate 046E or Moraxella catarrhalis isolate 56 and SEQ ID NO:4 is further defined as an amino acid sequence comprised within a CopB polypeptide of Moraxella catarrhalis isolate 4608.1.

60. The immunogenic composition according to claim 57, wherein the fragments are conjugated to a carrier protein.

61. The immunogenic composition according to claim 57, further comprising one or more adjuvants.

62. The immunogenic composition according to claim 57 further comprising additional antigens.

63. An immunogenic composition comprising a Moraxella catarrhalis polypeptide, wherein the polypeptide comprises at least the amino acid sequence of SEQ ID NO:3.

64. The immunogenic composition according to claim 63, wherein the polypeptide is further defined as a CopB polypeptide.

65. The immunogenic composition according to claim 63, wherein SEQ ID NO:3 is further defined as an amino acid sequence comprised within a CopB polypeptide of Moraxella catarrhalis isolate 430:345, Moraxelia catarrhalis isolate 046E or Moraxella catarrhalis isolate 56.

66. The immunogenic composition according to claim 63, wherein the polypeptide is conjugated to a carrier protein.

67. The immunogenic composition according to claim 63, further comprising one or more adjuvants.

68. The immunogenic composition according to claim 63, further comprising additional antigens.

69. An immunogenic composition comprising a Moraxella catarrhalis polypeptide, wherein the polypeptide comprises at least the amino acid sequence of SEQ ID NO:4.

70. The immunogenic composition according to claim 69, wherein the polypeptide is further defined as a CopB polypeptide.

71. The immunogenic composition of claim 69, wherein SEQ ID NO:4 is further defined as an amino acid sequence comprised within a CopB polypeptide of Moraxella catarrhalis isolate 4608.1.

72. The immunogenic composition according to claim 69, wherein the polypeptide is conjugated to a carrier protein.

73. The immunogenic composition according to claim 69, further comprising one or more adjuvants.

74. The immunogenic composition according to claim 69, further comprising additional antigens.

75. An immunogenic composition comprising at least two Moraxelia catarrhaiis polypeptides, wherein the first polypeptide comprises at least the amino acid sequence of SEQ ID NO:3 and the second polypeptide comprises at least the amino acid sequence of SEQ ID NO:4.

76. The immunogenic composition according to claim 75, wherein the polypeptides are further defined as CopB polypeptides.

77. The immunogenic composition of claim 75, wherein SEQ ID NO:3 is further defined as an amino acid sequence comprised within a CopB polypeptide of Moraxella catarrhalis isolate 430:345, Moraxella catarrhalls isolate 046E or Moraxella catarrhalis isolate 56 and SEQ ID NO:4 is further defined as an amino acid sequence comprised within a CopB polypeptide of Moraxella catarrhalis isolate 4608.1.

78. The immunogenic composition according to claim 75, wherein the polypeptides are conjugated to a carrier protein.

79. The immunogenic composition according to claim 75, further comprising one or more adjuvants.

80. The immunogenic composition according to claim 75, further comprising additional antigens.

81. An isolated polypeptide comprising a plurality of covalently linked Moraxella catarrhalis CopB epitope fragments, wherein the polypeptide comprises at least a fragment comprising an amino acid sequence of SEQ ID NO:3 and a fragment comprising an amino acid sequence of SEQ ID NO:4.

82. An isolated polypeptide comprising a plurality of covalently linked Moraxella catarrhalis CopB epitope fragments, wherein the polypeptide comprises at least a fragment comprising an amino acid sequence of SEQ ID NO:1, a fragment comprising an amino acid sequence of SEQ ID NO:2 and a fragment comprising an amino acid sequence of SEQ ID NO:3.

83. An isolated polypeptide comprising a plurality of covalently linked Moraxella cataffhalis CopB epitope fragments, wherein the polypeptide comprises at least a fragment comprising an amino acid sequence of SEQ ID NO:1, a fragment comprising an amino acid sequence of SEQ ID NO:2 and a fragment comprising an amino acid sequence of SEQ ID NO:4.

84. An isolated polypeptide comprising a plurality of covalently linked Moraxella catarrhalis CopB epitope fragments, wherein the polypeptide comprises at least a fragment comprising an amino acid sequence of SEQ ID NO:1, a fragment comprising an amino acid sequence of SEQ ID NO:2, a fragment comprising an amino acid sequence of SEQ ID NO:3 and a fragment comprising an amino acid sequence of SEQ ID NO:4.

85. An isolated polypeptide comprising a plurality of covalently linked Moraxella catarrhalis CopB epitope fragments of SEQ ID NO:3.

86. An isolated polypeptide comprising a plurality of covalently linked Moraxella catarrhalis CopB epitope fragments of SEQ ID NO:4.

87. An immunogenic composition comprising the polypeptide of claim 81.

88. The immunogenic composition of claim 87, further comprising one or more adjuvants.

89. The immunogenic composition according to claim 87, further comprising additional antigens.

90. An immunogenic composition comprising the polypeptide of claim 82.

91. The immunogenic composition of claim 90, further comprising one or more adjuvants.

92. The immunogenic composition according to claim 90, further comprising additional antigens.

93. An immunogenic composition comprising the polypeptide of claim 83.

94. The immunogenic composition of claim 93, further comprising one or more adjuvants.

95. The immunogenic composition according to claim 93, further comprising additional antigens.

96. An immunogenic composition comprising the polypeptide of claim 84.

97. The immunogenic composition of claim 96, further comprising one or more adjuvants.

98. The immunogenic composition according to claim 96, further comprising additional antigens.

99. An immunogenic composition comprising the polypeptide of claim 85.

100. The immunogenic composition of claim 99, further comprising one or more adjuvants.

101. The immunogenic composition according to claim 99, further comprising additional antigens.

102. An immunogenic composition comprising the polypeptide of claim 86.

103. The immunogenic composition of claim 102, further comprising one or more adjuvants.

104. The immunogenic composition according to claim 102, further comprising additional antigens.

105. A polynucleotide encoding a polypeptide of claim 81.

106. A polynucleotide encoding a polypeptide of claim 82.

107. A polynucleotide encoding a polypeptide of claim 83.

108. A polynucleotide encoding a polypeptide of claim 84.

109. A polynucleotide encoding a polypeptide of claim 85.

110. A polynucleotide encoding a polypeptide of claim 86.

111. An expression vector comprising a polynucleotide of claim 105.

112. An expression vector comprising a polynucleotide of claim 106.

113. An expression vector comprising a polynucleotide of claim 107.

114. An expression vector comprising a polynucleotide of claim 108.

115. An expression vector comprising a polynucleotide of claim 109.

116. An expression vector comprising a polynucleotide of claim 110.

117. A host cell transformed, transfected or infected with the vector of claim 111.

118. A host cell transformed, transfected or infected with the vector of claim 112.

119. A host cell transformed, transfected or infected with the vector of claim 113.

120. A host cell transformed, transfected or infected with the vector of claim 114.

121. A host cell transformed, transfected or infected with the vector of claim 115.

122. A host cell transformed, transfected or infected with the vector of claim 116.

123. A method of immunizing a host against Moraxella catarrhalis infection, the method comprising administering to the host an immunogenic composition comprising Moraxella catarrhalis polypeptide fragments, wherein the fragments comprise amino acid sequences of SEQ ID NO:1, SEQ ID NO:2 and SEQ ID NO:3.

124. A method of immunizing a host against Moraxella catarrhalis infection, the method comprising administering to the host an immunogenic composition comprising Moraxella catarrhalis polypeptide fragments, wherein the fragments comprise amino acid sequences of SEQ ID NO:1, SEQ ID NO:2 and SEQ ID NO:4.

125. A method of immunizing a host against Moraxella catarrhalis infection, the method comprising administering to the host an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:3.

126. A method of immunizing a host against Moraxella catarrhalis infection, the method comprising administering to the host an immunogenic composition comprising at least three Moraxella catarrhalis polypeptides, wherein the first polypeptide comprises at least the amino acid sequence of SEQ ID NO:1, the second polypeptide comprises at least the amino acid sequence of SEQ ID NO:2 and the third polypeptide comprises at least the amino acid sequence of SEQ ID NO:4.

127. A method of immunizing a host against Moraxella catarrhalis infection, the method comprising administering to the host an immunogenic composition comprising a plurality of covalently linked Moraxella catarrhalis CopB epitope fragments, wherein the plurality comprises at least a fragment comprising an amino acid sequence of SEQ ID NO:1, a fragment comprising an amino acid sequence of SEQ ID NO:2 and a fragment comprising an amino acid sequence of SEQ ID NO:3.

128. A method of immunizing a host against Moraxella catarrhalis infection, the method comprising administering to the host, an immunogenic composition comprising a plurality of covalently linked Moraxella catarrhalis CopB epitope fragments, wherein the plurality comprises at least a fragment comprising an amino acid sequence of SEQ ID NO:1, a fragment comprising an amino acid sequence of SEQ ID NO:2 and a fragment comprising an amino acid sequence of SEQ ID NO:4.

129. The method according to claim 123, wherein the polypeptide is conjugated to an antigen carrier protein.

130. The method according to claim 124, wherein the polypeptide is conjugated to an antigen carrier protein.

131. The method according to claim 125, wherein the polypeptide is conjugated to an antigen carrier protein.

132. The method according to claim 126, wherein the polypeptide is conjugated to an antigen carrier protein.

133. The method according to claim 127, wherein the polypeptide is conjugated to an antigen carrier protein.

134. The method according to claim 128, wherein the polypeptide is conjugated to an antigen carrier protein.

135. The method according to claim 123, further comprising additional antigens.

136. The method according to claim 124, further comprising additional antigens.

137. The method according to claim 125, further comprising additional antigens.

138. The method according to claim 126, further comprising additional antigens.

139. The method according to claim 127, further comprising additional antigens.

140. The method according to claim 128, further comprising additional antigens.

141. The method according to claim 123, further comprising one or more adjuvants.

142. The method according to claim 124, further comprising one or more adjuvants.

143. The method according to claim 125, further comprising one or more adjuvants.

144. The method according to claim 126, further comprising one or more adjuvants.

145. The method according to claim 127, further comprising one or more adjuvants.

146. The method according to claim 128, further comprising one or more adjuvants.

147. A method of immunizing a host against Moraxella catarrhalis infection, the method comprising administering to the host an immunogenic composition comprising Moraxella catarrhalis polypeptide fragments, wherein the fragments comprise amino acid sequences of SEQ ID NO:1, SEQ ID NO:2, SEQ ID NO:3 and SEQ ID NO:4.

148. A method of immunizing a host against Moraxella catarrhalis infection,- the method comprising administering to the host an immunogenic composition comprising at least four Moraxella catarrhalis polypeptides, wherein the first polypeptide comprises at least the amino acid sequence of SEQ ID NO:1, the second polypeptide comprises at least the amino acid sequence of SEQ ID NO:2, the third polypeptide comprises at least the amino acid sequence of SEQ ID NO:3 and the fourth polypeptide comprises at least the amino acid sequence of SEQ ID NO:4.

149. A method of immunizing a host against Moraxella catarrhalis infection, the method comprising administering to the host an immunogenic composition comprising a plurality of covalently linked Moraxella catarrhalis CopB epitope fragments, wherein the plurality comprises at least a fragment comprising an amino acid sequence of SEQ ID NO:1, a fragment comprising an amino acid sequence of SEQ ID NO:2; a fragment comprising an amino acid sequence of SEQ ID NO:3 and a fragment comprising an amino acid sequence of SEQ ID NO:4.

150. The method according to claim 147, wherein the polypeptide is conjugated to an antigen carrier protein.

151. The method according to claim 148, wherein the polypeptide is conjugated to an antigen carrier protein.

152. The method according to claim 149, wherein the polypeptide is conjugated to an antigen carrier protein.

153. The method according to claim 147, further comprising additional antigens.

154. The method according to claim 148, further comprising additional antigens.

155. The method according to claim 149, further comprising additional antigens.

156. The method according to claim 147, further comprising one or more adjuvants.

157. The method according to claim 148, further comprising one or more adjuvants.

158. The method according to claim 149, further comprising one or more adjuvants.
